(19) United States
(12) Patent Application Publication
(10) Pub. No.: US 2026/0358339 A1
(43) Pub. Date: Nov. 12, 2026

(54) Methods and Compositions For Identifying Monocyte Subsets in A Sample

(71) Applicant: August SpinCo Corporation, Milford, MA (US)

(72) Inventor: Wei Huang, Cupertino, CA (US)

(21) Appl. No.: 19/739,960

(22) Filed: Jul. 13, 2026

Related U.S. Application Data
(63) Continuation of application No. 17/028,523, filed on Sep. 22, 2020.
(60) Provisional application No. 62/909,645, filed on Oct. 2, 2019.

(51) Int. Cl. G01N 33/569

(52) U.S. Cl. CPC G01N 33/56972; G01N 2800/52

(57) ABSTRACT

Methods of identifying monocyte subsets in a sample are provided. Aspects of the methods include assaying the sample to obtain relative expression level data for each of CD14, CD16, and CD192 (CCR2) and quantitative expression level data for HLA-DR; and employing the obtained data to identify monocyte subsets in the sample. Also provided are compositions and kits for practicing embodiments of the invention. The methods and compositions find use in a variety of different applications, including therapeutic applications.

### CROSS REFERENCE TO RELATED APPLICATION

Pursuant to 35 U.S.C. § 119(e), this application claims priority to the filing date of U.S. Provisional Patent Application Ser. No. 62/909,645, filed Oct. 2, 2019; the disclosure of which application is incorporated herein by reference.

### INTRODUCTION

Monocytes represent an important part of immune system and are involved in both adaptive and innate responses. In recent years, there is a growing interest in the study of monocytes for clinical diagnosis of diseases, such as cardiovascular disease and sepsis. Recently, there have been also reports in studying monocyte for the diagnosis of Chronic Myelomonocytic Leukemia (CMML), pre-eclampsia and in the predication of anti-PD-1 immunotherapy response.

Traditional study of monocytes as a whole population lacks clarity in clinical diagnosis. In the last few decades, more and more studies are focused on different subset of monocytes. Current consensus divides monocytes into classical (CD14++CD16−), intermediate (Cd14++CD16+) and non-classical (CD14+CD16+) subsets. A recent consensus paper published by European Society of Cardiology added CCR2 to help to distinguish intermediate and non-classical monocytes. HLA-DR has also been used as a biomarker to gate monocytes in flow cytometry in order to improve the specificity and consistency in the analysis of monocyte subsets.

Recently, there are reports that the frequency of CD14+HLA-DR^{lo/neg} monocyte population in total monocytes correlates with the overall immunosuppressive status of the patient and the efficacy of immunotherapy, including efficacy of checkpoint inhibitors (e.g. CTLA-4 and PD-1 inhibitors), cancer vaccines and potentially efficacy of Adoptive and Chimeric Antigen Receptor (CAR) T Cell Therapies (Mengos A E, Gastineau D A and Gustafson M P (2019) The CD14+HLA-DRlo/neg Monocyte: An Immunosuppressive Phenotype That Restrains Responses to Cancer Immunotherapy. Front. Immunol. 10:1147. doi: 10.3389/fimmu.2019.01147).

However, the gating and measuring of the CD14+HLA-DR^{lo/neg} monocytes is often subjective and inconsistent in the literature.

### SUMMARY

A better, quantitative way of measuring the same biomarker would be direct quantification of HLA-DR expression levels on each subset of monocytes, particularly the classical monocytes.

Methods of identifying monocyte subsets in a sample are provided. Aspects of the methods include assaying the sample to obtain relative expression level data for each of CD14, CD16, and CD192 (CCR2) and quantitative expression level data for HLA-DR; and employing the obtained data to identify monocyte subsets in the sample. Also provided are compositions and kits for practicing embodiments of the invention. The methods and compositions find use in a variety of different applications, including developing biomarkers for therapeutic applications.

### BRIEF DESCRIPTION OF THE FIGURES

FIG. 1 depicts a flow cytometer according to certain embodiments.

FIG. 2 provides an example of gating for monocyte subsets in whole blood analyzed with a four-reagent panel.

FIG. 3 shows HLA-DR expression for monocyte subsets in antibody bound per cell units (ABC).

FIG. 4 provides an example of gating for a cellular count calibration standard.

FIG. 5 shows exemplary gating for monocyte subset analysis in whole blood using a four-reagent panel.

FIG. 6 shows flow cytometric data for an HLA-DR expression level quantitation standard.

FIG. 7 shows calibration using an HLA-DR expression level quantitation standard.

FIG. 8 shows exemplary gating for monocyte subset analysis in whole blood using a four-reagent panel.

FIGS. 9-23 show gating for monocyte subsets and HLA-DR analysis.

### DETAILED DESCRIPTION

Methods of identifying monocyte subsets in a sample are provided. Aspects of the methods include assaying the sample to obtain relative expression level data for each of CD14, CD16, and CD192 (CCR2) and quantitative expression level data for HLA-DR; and employing the obtained data to identify monocyte subsets in the sample. Also provided are compositions and kits for practicing embodiments of the invention. The methods and compositions find use in a variety of different applications, including therapeutic applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, embodiments of methods will be described first in greater detail, followed by a description of embodiments of compositions and kits that find use in practicing the methods.

### Methods

As summarized above, methods for identifying monocyte subsets in a sample are provided. Methods according to embodiments of the invention may include distinguishing one or more monocyte subsets from one another in a sample. In some cases, the methods include obtaining a relative amount, e.g., percentage, of each of one or more monocyte subsets in a sample. In some cases, the methods include obtaining the absolute cell count (e.g., cells/μL) for one or more monocyte subsets in a sample. In some cases, the methods include obtaining expression level data for one or more markers and determining the identity of the monocyte subset based on the obtained expression level data. The obtained expression level data may include relative expression level data, e.g., expression levels of a marker compared to a control, for one or more markers and/or quantitative expression level data, e.g., quantity of marker that is expressed by a cell, for one or more markers.

In practicing embodiments of the methods, aspects of the methods include assaying the sample to obtain relative expression level data for each of CD14, CD16, and CD192 (CCR2) and quantitative expression level data for HLA-DR; and employing the obtained data to identify monocyte subsets in the sample. “Expression level” as used herein is employed in its conventional sense to refer to the relative or absolute amount of mRNA and/or protein expressed by a cell, e.g., inside the cell or on the surface of the cell.

As summarized above, aspects of the subject methods may include assaying the sample to obtain relative expression level data for each of CD14, CD16, and CD192 (CCR2) and quantitative expression level data for HLA-DR. The relative expression level data for each of CD14, CD16, and CD192 may be obtained for each monocyte or monocyte subset in a sample. The relative expression level data may include expression levels of each of CD14, CD16, and CD192 relative to a control, e.g., a positive or negative control. In some cases, the relative expression level data indicates whether the monocyte subset expresses one or more of CD14, CD16, and CD192, e.g., whether the monocyte subset is positive or negative for one or more of the markers. In some cases, the relative expression level data indicates whether the monocyte subset expresses a low or high amount of one or more of CD14, CD16, and CD192 compared to a control, e.g., a control with a low or high amount of a marker.

The quantitative expression level data for HLA-DR may include the absolute amount of HLA-DR expressed by a cell, e.g., on a surface of the cell. In certain embodiments, the quantitative expression level data may include the number of molecules of HLA-DR expressed by the cell. In some cases, the quantitative expression level data for HLA-DR is obtained for each of the monocyte subsets in a sample, e.g., each of the identified monocyte subsets in the sample. The quantitative expression level data for HLA-DR may be obtained with use of an HLA-DR expression level quantitation standard as described in detail below.

Aspects of the methods may further include employing the obtained data, e.g., flow cytometric data, to identify monocyte subsets in the sample. The relative expression level data and/or the quantitative expression level data, e.g., obtained from flow cytometric analysis of a sample, may make up a marker expression profile that distinguishes the monocyte subset. The obtained data may be employed to identify monocyte subsets including, e.g., classical (M1), Intermediate (M2), and non-classical (M3). Classical monocytes may include any one of CD14+CD16lo cells, CD14+CD16− cells, CD14++CD16− cells, CD14highCD16− cells, CD14++CD16−CCR2+. Intermediate monocytes may include any one of CD14++CD16+ cells, CD14++CD16+CCR2+ cells, and CD14+CD16+CCR2 hi cells. Nonclassical monocytes may include any one of CD14+CD16+ cells, CD14+CD16++ cells, CD14+CD16+CCR2lo cells, and CD14+CD16++CCR2− cells. In certain embodiments, the obtained data is used to calculate the percentage of each monocyte subset. In certain embodiments, the methods provide the absolute cell count for each monocyte subset in the sample.

### Preparing a Labeled Sample

In practicing embodiments of the subject methods, the assaying of a sample may include contacting the sample with distinguishably fluorescently labeled specific binding members for each of CD14, CD16, CD192 (CCR2) and HLA-DR to produce a labeled sample. The term “binding member” as used herein refers to any agent (e.g., a protein (e.g., antibody or binding fragment thereof), aptamer, small molecule, and the like) that specifically binds to a target analyte. The terms “specific binding,” “specifically binds,” and the like, refer to the preferential binding to a molecule relative to other molecules or moieties in a solution or reaction mixture. In some embodiments, the affinity between a binding member and the target analyte (e.g., a marker as described above) to which it specifically binds when they are specifically bound to each other in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower K_{d}.

In certain aspects, the specific binding reagent is an antibody, or antigen-binding fragment thereof. As used herein, the term “antibodies” includes antibodies or immunoglobulins of any isotype, fragments of antibodies which retain specific binding to an antigen, including, but not limited to, Fab, Fv, scFv, and Fd fragments, chimeric antibodies, humanized antibodies, fully human antibodies, single-chain antibodies, and fusion proteins comprising an antigen-binding portion of an antibody and a non-antibody protein. The antibodies may be further conjugated to other moieties, such as members of specific binding pairs, e.g., biotin (member of biotin-avidin specific binding pair), and the like. Also encompassed by the term are Fab′, Fv, F(ab′)₂, and other antibody fragments that retain specific binding to an antigen, and monoclonal antibodies. In other aspects, the binding members may be antigens, where the analytes of interest are antibodies.

The specific binding members may be distinguishably fluorescently labeled. By “distinguishably fluorescently labeled” is meant each of the specific binding members used in the methods is coupled to a fluorescent label that is distinct (e.g., absorbs or emits a different wavelength) from the fluorescent label of another specific binding member used in the methods. Suitable labels may include detectable moieties or markers that are detectible based on, for example, fluorescence emission, absorbance, fluorescence polarization, fluorescence lifetime, fluorescence wavelength, absorbance wavelength, Stokes shift, light scatter, mass, molecular mass, redox, acoustic, raman, magnetism, radio frequency, enzymatic reactions (including chemiluminescence and electro-chemiluminescence) or combinations thereof. For example, the label may be a fluorophore, chromophore, enzyme, redox label, radio label, acoustic label, Raman (SERS) tag, mass tag, isotope tag (e.g., isotopically pure rare earth element), magnetic particle, microparticle as well as a nanoparticle. In certain embodiments, the label is a fluorophore (i.e., a fluorescent label, fluorescent dye, etc.). Fluorophores of interest may include but are not limited to dyes suitable for use in analytical applications (e.g., flow cytometry, imaging, etc.), such as an acridine dye, anthraquinone dyes, arylmethane dyes, diarylmethane dyes (e.g., diphenyl methane dyes), chlorophyll containing dyes, triarylmethane dyes (e.g., triphenylmethane dyes), azo dyes, diazonium dyes, nitro dyes, nitroso dyes, phthalocyanine dyes, cyanine dyes, asymmetric cyanine dyes, quinon-imine dyes, azine dyes, eurhodin dyes, safranin dyes, indamins, indophenol dyes, fluorine dyes, oxazine dye, oxazone dyes, thiazine dyes, thiazole dyes, xanthene dyes, fluorene dyes, pyronin dyes, fluorine dyes, rhodamine dyes, phenanthridine dyes, as well as dyes combining two or more of the aforementioned dyes (e.g., in tandem), polymeric dyes having one or more monomeric dye units and mixtures of two or more of the aforementioned dyes thereof. A large number of dyes are commercially available from a variety of sources, such as, for example, Molecular Probes (Eugene, OR), Dyomics GmbH (Jena, Germany), Sigma-Aldrich (St. Louis, MO), Sirigen, Inc. (Santa Barbara, CA) and Exciton (Dayton, OH). For example, the fluorophore may include 4-acetamido-4′-isothiocyanatostilbene-2,2′disulfonic acid; acridine and derivatives such as acridine, acridine orange, acridine yellow, acridine red, and acridine isothiocyanate; allophycocyanin, phycoerythrin, peridinin-chlorophyll protein, 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy3.5, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; dye-conjugated polymers (i.e., polymer-attached dyes) such as fluorescein isothiocyanate-dextran as well as dyes combining two or more dyes (e.g., in tandem), polymeric dyes having one or more monomeric dye units and mixtures of two or more of the aforementioned dyes or combinations thereof.

In some instances, the fluorophore (i.e., dye) is a fluorescent polymeric dye. Fluorescent polymeric dyes that find use in the subject methods and systems are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where rr-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. As summarized above, the structural characteristics of the polymeric dyes can have an effect on the fluorescence properties of the molecules. Any convenient polymeric dye may be utilized in the subject methods and systems. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light. The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via energy transfer. In some instances, the polymer may be used as a direct fluorescent reporter, for example fluorescent polymers having high extinction coefficients, high brightness, etc. In some instances, the polymer may be used as a strong chromophore where the color or optical density is used as an indicator. Polymeric dyes of interest include, but are not limited to, those dyes described by Gaylord et al. in US Publication Nos. 20040142344, 20080293164, 20080064042, 20100136702, 20110256549, 20120028828, 20120252986, 20130190193 and 20160025735 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety. Specific polymeric dyes that may be employed include, but are not limited to, BD Horizon Brilliant™ Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515).

Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc.

In certain embodiments, the labeled sample is produced by combining the sample, e.g., whole blood sample, with the distinguishably fluorescently labeled specific binding members in a container. In certain embodiments, the methods include introducing the sample into a container holding the distinguishably labeled specific binding members to promote contact of the sample and the labeled specific binding members. The distinguishably fluorescently labeled specific binding members may be present in a composition, e.g., a solid or liquid composition. In certain embodiments, the composition is a dried composition, as described in detail below. The introduction of the sample into the container may reconstitute the dried composition.

In certain cases, the contacting may be carried out in a binding buffer. The binding buffer may include molecules standard for antigen-antibody binding buffers such as, albumin (e.g., BSA), non-ionic detergents (Tween-20, Triton X-100), and/or protease inhibitors (e.g., PMSF). In certain cases, the binding buffer may be disposed in the container prior to or after adding the whole blood sample to the container. In certain cases, the container may already contain the leukocyte specific binding reagent and binding buffer before a whole blood sample is added to the container. The term “incubating” is used synonymously with “contacting” and “exposing” and does not imply any specific time or temperature requirements unless otherwise indicated.

Where desired, lysing the RBCs of the sample via a RBC lysis reagent is carried out to provide a RBC depleted sample. The lysing step may include contacting the sample with a RBC lysis reagent. It is noted that in certain cases, the sample is not removed from the container in which the sample, e.g., whole blood sample, was contacted with the binding members. The RBC lysis reagent may be a glycoside, such as, saponin; a hypotonic solution of an ammonium salt, e.g., ammonium chloride; an enzyme that causes lysis of the cell wall of RBCs, e.g., hemolysin; or a detergent, e.g., an ionic or a non-ionic detergent. Numerous standard RBC lysis reagents are available. For example, the RBC lysis reagent may be obtained from a commercial supplier. The concentration of a RBC lysis agent, as well as a lysis buffer, if used, may be adjusted for optimal results. When a RBC lysis agent, and an optional lysis buffer, is present at lower concentrations, RBC cell lysis may be suboptimal. At higher concentrations, undesirable cellular disruption may occur. Routine empirical approaches can be carried out to determine the preferred concentration. When the RBC lysis reagent is from a commercial source, the manufacturer's protocol may be followed and further optimized, if needed. In certain cases, the RBC lysis reagent may be saponin and the saponin may be present at a concentration of 0.03%-3% (w/v).

In certain embodiments, the methods may further include combining the sample, e.g., labeled sample, with a cellular count calibration standard. In some instances, the methods include combining, in any suitable order, an amount of sample with one or more labeled specific binding members, e.g., distinguishably fluorescently labeled specific binding members, as described above, and a cellular count calibration standard. In some instances, the methods include combing an amount of prepared labeled sample, as described above, with the cellular count calibration standard. The labeled sample including the cellular count calibration standard may be used to determine the cell count for one or more monocyte subsets, as described in detail below. The cellular count calibration standard may include a known count or quantity of particles. In some cases, the cellular count calibration standard is a dried composition including detectible particles, e.g., fluorescently labeled beads. In some embodiments, the cellular count calibration standard includes a BD Trucount™ bead composition.

Contacting the sample may be achieved using any convenient protocol, including stirring, agitation, etc. The contacting of the sample with each of the fluorescently labeled specific binding members may occur simultaneously or sequentially. In certain embodiments, the contacting occurs by incubating the sample with the fluorescently labeled specific binding members. In certain embodiments, the contacting includes mixing the sample and the fluorescently labeled binding members. The mixing may be performed using an agitator. The agitator may be any convenient agitator sufficient for mixing the liquid inside a liquid container, including, but not limited to, vortexers, sonicators, shakers (e.g., manual, mechanical, or electrically powered shakers), rockers, oscillating plates, magnetic stirrers, static mixers, rotators, blenders, mixers, tumblers, orbital shakers, among other agitating protocols. The contacting may occur at a variety of temperatures, where the temperature ranges in some instances from 16 to 30 °C, such as 20 to 25 °C. The contacting may occur for any suitable amount of time where the amount of time ranges in some instances from 1 minute to 30 minutes, from 30 minutes to 1 hour, from 30 minutes to 2 hours, from 30 minutes to 5 hours, from 30 minutes to 8 hours, from 1 day to 2 days, from 1 day to 5 days, or 1 day to 7 days.

In certain embodiments, the method further includes obtaining the sample from a subject, e.g., before contacting the sample with the fluorescently labeled specific binding members. The sample may be obtained by any convenient means. In some instances, the sample is obtained under sterile conditions. The sample may vary. A “biological sample” encompasses a variety of sample types obtained from an individual. The definition encompasses biological fluids (e.g., blood (including blood fractions (e.g., serum, plasma)); and other liquid samples of biological origin (e.g., saliva, urine, bile fluid), as well as solid tissue samples in the form of a liver biopsy specimen. Sample sources of interest include, but are not limited to, CSF, urine, saliva, tears, tissue derived samples, e.g. homogenates, and blood or derivatives thereof. “Blood sample” refers to a biological sample, which is obtained from blood of a subject, and includes whole blood and blood fractions (e.g., plasma or serum) suitable for analysis in the present methods. In general, separation of cellular components and non-cellular components in a blood sample (e.g., by centrifugation) without coagulation provides a blood plasma sample, while such separation of coagulated (clotted) blood provides a blood serum sample. Examples of biological samples of blood include peripheral blood or samples derived from peripheral blood. The definition also includes samples that have been manipulated after their procurement, such as by treatment with reagents, solubilization, or enrichment for certain components, such as one or more polypeptides to be assayed. For example, a biological sample (e.g., blood) can be enriched for a fraction containing an analyte(s) of interest. Where the sample is a blood sample, the sample may be obtained by any convenient means including, e.g., by finger stick, heel stick, or venipuncture. Blood samples may also be taken from patients by venous or arterial lines.

In some instances, the sample is a whole blood sample. As used herein, “whole blood” refers to blood from which no constituent, such as red blood cells, white blood cells, plasma, or platelets, has been removed. Whole blood sample refers to a sample of whole blood collected from a subject. In some instances, the sample may be blood fraction, e.g., a blood sample from which one or more constituents, e.g., red bloods cells, has been removed. The terms “subject”, “individual”, and “patient” are used herein interchangeably to refer to the subject from whom the sample, e.g., whole blood sample or blood fraction sample, has been obtained.

In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

### Flow Cytometric Analysis

In practicing embodiments of the methods, the assaying may further include flow cytometrically analyzing the labeled sample and an HLA-DR expression level quantitation standard to obtain fluorescence intensity level data for each of CD14, CD16, CD192 and HLA-DR, as well as quantitative expression level data for HLA-DR. The fluorescence intensity level data and/or the quantitative expression level data may be used to identify the monocyte subsets in a sample. In some instances, relative expression level data may be derived from the fluorescence intensity level data. For example, the fluorescence intensity level data may indicate whether a subpopulation of cells expresses one or more markers or a high or low amount of one or more markers.

The flow cytometric analysis of the labeled sample may include performing a flow cytometric assay on the labeled sample. The flow cytometric analysis may include characterizing the components in a labeled sample with a flow cytometer system. The flow cytometric analysis may include introducing the labeled sample into a flow cytometer. The flow cytometric assaying may include introducing the labeled sample into a flow cytometer. A flow cytometer typically includes a sample reservoir for receiving a fluid sample, such as the labeled sample, and a sheath reservoir containing a sheath fluid. The flow cytometer transports the particles (including cells, e.g., from the labeled sample) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of detectors, one for each of the scatter parameters to be measured, and one or more for each of the distinct dyes to be detected are included in the analyzer. For example, some embodiments include spectral configurations where more than one sensor or detector is used per dye. The data obtained include the signals measured for each of the light scatter detectors and the fluorescence emissions.

As summarized above, in detecting, counting and/or sorting analytes, a liquid medium comprising the analytes, e.g., labeled sample, is introduced into the flow path of a flow cytometer. When in the flow path, the analytes are passed substantially one at a time through one or more sensing regions (e.g., an interrogation point), where each of the analytes is exposed individually to a source of light at a single wavelength and measurements of light scatter parameters and/or fluorescent emissions as desired (e.g., two or more light scatter parameters and measurements of one or more fluorescent emissions) are separately recorded for each analyte. The data recorded for each analyte is analyzed in real time or stored in a data storage and analysis means, such as a computer, as desired. U.S. Pat. No. 4,284,412 describes the configuration and use of a typical flow cytometer equipped with a single light source, while U.S. Pat. No. 4,727,020 describes the configuration and use of a flow cytometer equipped with two light sources. The disclosures of these patents are herein incorporated by reference in their entireties for all purposes. Flow cytometers having more than two light sources may also be employed.

More specifically, in a flow cytometer, the analytes are passed, in suspension, substantially one at a time in a flow path through one or more sensing regions (or “interrogation points”) where in each region each analyte is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 635 nm, and the like.

In certain embodiments, a sample (e.g., in a flow stream of the flow cytometer) may be irradiated with light from a light source. In series with a sensing region, detectors, e.g., light collectors, such as photomultiplier tubes (or “PMT”), are used to record light that passes through each analyte (generally referred to as forward light scatter), light that is reflected orthogonal to the direction of the flow of the analytes through the sensing region (generally referred to as orthogonal or side light scatter) and fluorescent light emitted from the labeled analyte, as the analyte passes through the sensing region and is illuminated by the energy source. Each of forward light scatter (or FSC), orthogonal light scatter (SSC), and fluorescence emissions (FL1, FL2, etc.) comprise a separate parameter for each analyte (or each “event”). Thus, for example, two, three or four parameters can be collected (and recorded) from an analyte labeled with two different fluorescent labels.

In other embodiments, methods include irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

In certain embodiments, methods include irradiating the sample with one or more lasers. As discussed above, the type and number of lasers will vary depending on the sample as well as desired light collected and may be a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCI) excimer laser or xenon-fluorine (XeF) excimer laser or a combination thereof. In others instances, the methods include irradiating the flow stream with a dye laser, such as a stilbene, coumarin or rhodamine laser. In yet other instances, methods include irradiating the flow stream with a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, methods include irradiating the flow stream with a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

The sample may be irradiated with one or more of the above mentioned light sources, such as 2 or more light sources, such as 3 or more light sources, such as 4 or more light sources, such as 5 or more light sources and including 10 or more light sources. The light source may include any combination of types of light sources. For example, in some embodiments, the methods include irradiating the sample in the flow stream with an array of lasers, such as an array having one or more gas lasers, one or more dye lasers and one or more solid-state lasers.

In certain instances, the flow stream is irradiated with a plurality of beams of frequency-shifted light and a cell in the flow stream is imaged by fluorescence imaging using radiofrequency tagged emission (FIRE) to generate a frequency-encoded image, such as those described in Diebold, et al. Nature Photonics Vol. 7(10); 806-810 (2013) as well as described in U.S. Pat. Nos. 9,423,353; 9,784,661 and 10,006,852 and U.S. Patent Publication Nos. 2017/0133857 and 2017/0350803, the disclosures of which are herein incorporated by reference.

Accordingly, in flow cytometrically assaying the analytes, the analytes may be detected and uniquely identified by exposing the particles to excitation light and measuring the fluorescence of each analyte in one or more detection channels, as desired. The excitation light may be from one or more light sources and may be either narrow or broadband. Examples of excitation light sources include lasers, light emitting diodes, and arc lamps. Fluorescence emitted in detection channels used to identify the analytes may be measured following excitation with a single light source, or may be measured separately following excitation with distinct light sources. If separate excitation light sources are used to excite the fluorescent labels, the labels may be selected such that all the labels are excitable by each of the excitation light sources used.

Aspects of the present methods include collecting fluorescent light with a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescent light detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescent light detectors. In embodiments, each of the fluorescent light detectors is configured to generate a fluorescence data signal. Fluorescence from the sample may be detected by each fluorescent light detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescent light detectors in the subject light detection system. In certain embodiments, methods include detecting wavelengths of light which correspond to the fluorescence peak wavelength of certain fluorophores present in the sample. In embodiments, fluorescent flow cytometer data is received from one or more fluorescent light detectors (e.g., one or more detection channels), such as 2 or more, such as 3 or more, such as 4 or more, such as 5 or more, such as 6 or more and including 8 or more fluorescent light detectors (e.g., 8 or more detection channels).

Light from the sample may be measured at one or more wavelengths of, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring the collected light at 400 or more different wavelengths.

In certain embodiments, methods include spectrally resolving the light from each fluorophore of the fluorophore-biomolecule reagent pairs in the sample. In some embodiments, the overlap between each different fluorophore is determined and the contribution of each fluorophore to the overlapping fluorescence is calculated. In some embodiments, spectrally resolving light from each fluorophore includes calculating a spectral unmixing matrix for the fluorescence spectra for each of the plurality of fluorophores having overlapping fluorescence in the sample detected by the light detection system. In certain instances, spectrally resolving the light from each fluorophore and calculating a spectral unmixing matrix for each fluorophore may be used to estimate the abundance of each fluorophore, such as for example to resolve the abundance of target cells in the sample.

In certain embodiments, methods include spectrally resolving light detected by a plurality of photodetectors such as described e.g., in International Patent Application No. PCT/US2019/068395 filed on Dec. 23, 2019; U.S. Provisional Patent Application No. 62/971,840 filed on Feb. 7, 2020 and U.S. Provisional Patent Application No. 63/010,890 filed on Apr. 16, 2020, the disclosures of which are herein incorporated by reference in their entirety. For example, spectrally resolving light detected by the plurality of photodetectors of the second set of photodetectors may be include solving a spectral unmixing matrix using one or more of: 1) a weighted least square algorithm; 2) a Sherman-Morrison iterative inverse updater; 3) an LU matrix decomposition, such as where a matrix is decomposed into a product of a lower-triangular (L) matrix and an upper-triangular (U) matrix; 4) a modified Cholesky decomposition; 5) by QR factorization; and 6) calculating a weighted least squares algorithm by singular value decomposition.

In certain embodiments, methods further include characterizing the spillover spreading of the light detected by a plurality of photodetectors such as described e.g., in U.S. Provisional Patent Application Nos. 63/020,758 and 63/076,611, the disclosures of which are herein incorporated by reference.

In certain instances, the abundance of fluorophores associated with (e.g., chemically associated (i.e., covalently, ionically) or physically associated) a target particle is calculated from the spectrally resolved light from each fluorophore associated with the particle. For instance, in one example the relative abundance of each fluorophore associated with a target particle is calculated from the spectrally resolved light from each fluorophore. In another example, the absolute abundance of each fluorophore associated with the target particle is calculated from the spectrally resolved light from each fluorophore. In certain embodiments, a particle may be identified or classified based on the relative abundance of each fluorophore determined to be associated with the particle. In these embodiments, the particle may be identified or classified by any convenient protocol such as by: comparing the relative or absolute abundance of each fluorophore associated with a particle with a control sample having particles of known identity; or by conducting spectroscopic or other assay analysis of a population of particles (e.g., cells) having the calculated relative or absolute abundance of associated fluorophores.

In certain embodiments, methods include sorting one or more of the particles (e.g., cells) of the sample that are identified based on the estimated abundance of the fluorophores associated with the particle. The term “sorting” is used herein in its conventional sense to refer to separating components (e.g., droplets containing cells, droplets containing non-cellular particles such as biological macromolecules) of a sample and in some instances, delivering the separated components to one or more sample collection containers. For example, methods may include sorting 2 or more components of the sample, such as 3 or more components, such as 4 or more components, such as 5 or more components, such as 10 or more components, such as 15 or more components and including sorting 25 or more components of the sample.

In sorting particles identified based on the abundance of fluorophores associated with the particle, methods include data acquisition, analysis and recording, such as with a computer, where multiple data channels record data from each detector used in obtaining the overlapping spectra of the plurality of fluorophore-biomolecule reagent pairs associated with the particle. In these embodiments, analysis includes spectrally resolving light (e.g., by calculating the spectral unmixing matrix) from the plurality of fluorophores of the fluorophore-biomolecule reagent pairs having overlapping spectra that are associated with the particle and identifying the particle based on the estimated abundance of each fluorophore associated with the particle. This analysis may be conveyed to a sorting system which is configured to generate a set of digitized parameters based on the particle classification.

In some embodiments, methods for sorting components of a sample include sorting particles (e.g., cells in a biological sample), such as described in U.S. Pat. Nos. 3,960,449; 4,347,935; 4,667,830; 5,245,318; 5,464,581; 5,483,469; 5,602,039; 5,643,796; 5,700,692; 6,372,506 and 6,809,804, the disclosures of which are herein incorporated by reference. In some embodiments, methods include sorting components of the sample with a particle sorting module, such as those described in U.S. Pat. Nos. 9,551,643 and 10,324,019, U.S. Patent Publication No. 2017/0299493 and International Patent Publication No. WO/2017/040151, the disclosure of which is incorporated herein by reference. In certain embodiments, cells of the sample are sorted using a sort decision module having a plurality of sort decision units, such as those described in U.S. patent application Ser. No. 16/725,756, filed on Dec. 23, 2019, the disclosure of which is incorporated herein by reference.

Flow cytometric assay procedures are well known in the art. See, e.g., Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49 (pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain aspects, flow cytometrically assaying the composition involves using a flow cytometer capable of simultaneous excitation and detection of multiple fluorophores, such as a BD Biosciences FACSCanto™ flow cytometer, used substantially according to the manufacturer's instructions. Methods of the present disclosure may involve image cytometry, such as is described in Holden et al. (2005) Nature Methods 2:773 and Valet, et al. 2004 Cytometry 59:167-171, the disclosures of which are incorporated herein by reference.

As discussed above, the method includes cytometric analysis which may include sorting. Cells of interest identified in the sample may be sorted and subsequently analyzed by any convenient analysis technique. Subsequent analysis techniques of interest include, but are not limited to, sequencing; assaying by CellSearch, as described in Food and Drug Administration (2004) Final rule. Fed Regist 69: 26036-26038; assaying by CTC Chip, as described in Nagrath, et al. (2007) Nature 450: 1235-1239; assaying by MagSweeper, as described in Talasaz, et al. (2009). Proc Natl Acad Sci USA 106: 3970-3975; and assaying by nanostructured substrates, as described in Wang S, et al. (2011) Angew Chem Int Ed Engl 50: 3084-3088; the disclosures of which are incorporated herein by reference. Where desired, the sorting protocol may include distinguishing viable and dead cells, where any convenient staining protocol for identifying such cells may be incorporated into the methods.

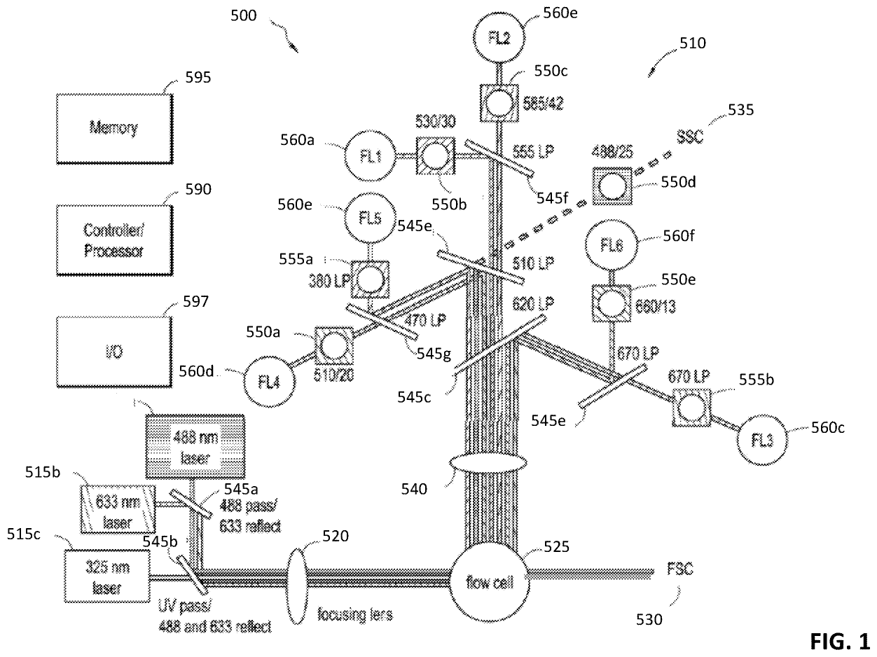

FIG. 1 shows a system 500 for flow cytometry in accordance with an illustrative embodiment of the present invention. The system 500 includes a flow cytometer 510, a controller/processor 590 and a memory 595. The flow cytometer 510 includes one or more excitation lasers 515a-515c, a focusing lens 520, a flow chamber 525, a forward scatter detector 530, a side scatter detector 535, a fluorescence collection lens 540, one or more beam splitters 545a-545g, one or more bandpass filters 550a-550e, one or more longpass (“LP”) filters 555a-555b, and one or more fluorescent detectors 560a-560f.

The excitation lasers 515a-c emit light in the form of a laser beam. The wavelengths of the laser beams emitted from excitation lasers 515a-515c may be 488 nm, 633 nm, and 325 nm, respectively. The laser beams are first directed through one or more of beam splitters 545a and 545b. Beam splitter 445a transmits light at 488 nm and reflects light at 633 nm. Beam splitter 545b transmits UV light (light with a wavelength in the range of 10 to 400 nm) and reflects light at 488 nm and 633 nm.

The laser beams are then directed to a focusing lens 520, which focuses the beams onto the portion of a fluid stream where particles of a sample are located, within the flow chamber 525. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward scatter detector 530, the side scatter detector 535, and the one or more fluorescent detectors 560a-560f through one or more of the beam splitters 545a-545g, the bandpass filters 550a-550e, the longpass filters 555a-555b, and the fluorescence collection lens 540.

The fluorescence collection lens 540 collects light emitted from the particle-laser beam interaction and routes that light towards one or more beam splitters and filters. Bandpass filters, such as bandpass filters 550a-550e, allow a narrow range of wavelengths to pass through the filter. For example, bandpass filter 550a is a 510/20 filter. The first number represents the center of a spectral band. The second number provides a range of the spectral band. Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 555a-555b, transmit wavelengths of light equal to or longer than a specified wavelength of light. For example, longpass filter 555a, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

Beam splitters direct light of different wavelengths in different directions. Beam splitters can be characterized by filter properties such as shortpass and longpass. For example, beam splitter 545g is a 620 SP beam splitter, meaning that the beam splitter 545g transmits wavelengths of light that are 620 nm or shorter and reflects wavelengths of light that are longer than 620 nm in a different direction. In one embodiment, the beam splitters 545a-545g can comprise optical mirrors, such as dichroic mirrors.

The forward scatter detector 530 is positioned slightly off axis from the direct beam through the flow cell and is configured to detect diffracted light, the excitation light that travels through or around the particle in mostly a forward direction. The intensity of the light detected by the forward scatter detector is dependent on the overall size of the particle. The forward scatter detector can include a photodiode. The side scatter detector 535 is configured to detect refracted and reflected light from the surfaces and internal structures of the particle, and tends to increase with increasing particle complexity of structure. The fluorescence emissions from fluorescent molecules associated with the particle can be detected by the one or more fluorescent detectors 560a-560f. The side scatter detector 535 and fluorescent detectors can include photomultiplier tubes. The signals detected at the forward scatter detector 530, the side scatter detector 535 and the fluorescent detectors can be converted to electronic signals (voltages) by the detectors. This data can provide information about the sample.

One of skill in the art will recognize that a flow cytometer in accordance with an embodiment of the present invention is not limited to the flow cytometer depicted in FIG. 1, but can include any flow cytometer known in the art. For example, a flow cytometer may have any number of lasers, beam splitters, filters, and detectors at various wavelengths and in various different configurations.

In operation, cytometer operation is controlled by a controller/processor 590, and the measurement data from the detectors can be stored in the memory 595 and processed by the controller/processor 590. Although not shown explicitly, the controller/processor 590 is coupled to the detectors to receive the output signals therefrom, and may also be coupled to electrical and electromechanical components of the flow cytometer 500 to control the lasers, fluid flow parameters, and the like. Input/output (I/O) capabilities 597 may be provided also in the system. The memory 595, controller/processor 590, and I/O 597 may be entirely provided as an integral part of the flow cytometer 510. In such an embodiment, a display may also form part of the I/O capabilities 597 for presenting experimental data to users of the cytometer 500. Alternatively, some or all of the memory 595 and controller/processor 590 and I/O capabilities may be part of one or more external devices such as a general purpose computer. In some embodiments, some or all of the memory 595 and controller/processor 590 can be in wireless or wired communication with the cytometer 510. The controller/processor 590 in conjunction with the memory 595 and the I/O 597 can be configured to perform various functions related to the preparation and analysis of a flow cytometer experiment.

The system illustrated in FIG. 1 includes six different detectors that detect fluorescent light in six different wavelength bands (which may be referred to herein as a “filter window” for a given detector) as defined by the configuration of filters and/or splitters in the beam path from the flow cell 525 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. However, as more detectors are provided, and more labels are utilized, perfect correspondence between filter windows and fluorescent emission spectra is not possible. It is generally true that although the peak of the emission spectra of a particular fluorescent molecule may lie within the filter window of one particular detector, some of the emission spectra of that label will also overlap the filter windows of one or more other detectors. This may be referred to as spillover. The I/O 597 can be configured to receive data regarding a flow cytometer experiment having a panel of fluorescent labels and a plurality of cell populations having a plurality of markers, each cell population having a subset of the plurality of markers. The I/O 597 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 595. The controller/processor 590 can be configured to evaluate one or more assignments of labels to markers.

Flow cytometers further include data acquisition, analysis and recording means, such as a computer, wherein multiple data channels record data from each detector for the light scatter and fluorescence emitted by each analyte as it passes through the sensing region. The purpose of the analysis system is to classify and count analytes where each analyte presents itself as a set of digitized parameter values. In flow cytometrically assaying (e.g., detecting, counting and/or sorting) particles in methods of the present disclosure, the flow cytometer may be set to trigger on a selected parameter in order to distinguish the analytes of interest from background and noise. “Trigger” refers to a preset threshold for detection of a parameter. It is typically used as a means for detecting passage of a particle through the laser beam. Detection of an event which exceeds the threshold for the selected parameter triggers acquisition of light scatter and fluorescence data for the analyte. Data is not acquired for analytes or other components in the sample being assayed which cause a response below the threshold. The trigger parameter may be the detection of forward scattered light caused by passage of an analyte through the light beam. The flow cytometer then detects and collects the light scatter and fluorescence data for the analyte.

A particular subpopulation of interest, e.g., a monocyte subset, is then further analyzed by “gating” based on the data collected for the entire population. In some instances, one or more of the labeled markers CD14, CD16, HLA-DR and CD129 may be used to gate each subset of monocytes. In some instances, the monocyte subsets may be gated based on the fluorescence intensity levels for the markers obtained from the flow cytometric analysis. In some instances, the monocyte subsets are further gated based on other parameters including, e.g., forward and side scatter. To select an appropriate gate, the data is plotted so as to obtain the best separation of subpopulations possible. This procedure is typically done by plotting a first parameter, e.g., forward light scatter (FSC) or light from a first label, vs. a second parameter, e.g., side (i.e., orthogonal) light scatter (SSC) or light from a second label, on a two-dimensional dot plot. The flow cytometer operator then selects the desired subpopulation of analytes (i.e., those cells within the gate) and excludes analytes which are not within the gate. Where desired, the operator may select the gate by drawing a line around the desired subpopulation using a cursor on a computer screen. Only those analytes within the gate are then further analyzed by plotting the other parameters for these analytes, such as fluorescence. A given protocol may include multiple gating steps, as desired.

Some flow cytometers are equipped to sort particles as they flow through the machine, redirecting the particle (after the particle has been interrogated/evaluated) to a particular location (e.g., into a desired sample collection container).

During sorting, the fluid stream is broken into highly uniform droplets, which detach from the stream. The time between when a particle intercepts the energy source (e.g., the laser) and when it reaches the droplet breakoff point is determined. When a particle is detected that meets the predefined sorting criteria, an electrical charge is applied to the stream just as the droplet containing that particle breaks off from the stream. Once broken off from the stream, the droplet—now surrounded by air—retains its charge. The charged droplet passes by two strongly charged deflection plates. Electrostatic attraction and repulsion cause each charged droplet to be deflected to the left or right, depending on the droplet's charge polarity. For example, in some cases, a flow cytometer can sort particles into one of two different tubes, or into a desired well of a multi-well plate (e.g., a 6-well plate, a 12-well plate, a 24-well plate, a 48-well plate, a 96-well plate, etc.). Uncharged droplets are not affected by the electric field and pass down the center to be collected or aspirated as waste.

Any convenient flow cytometer may be employed. In some embodiments, the subject systems are flow cytometric systems. Suitable flow cytometry systems may include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49 (pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ II flow cytometer, BD Accuri™ flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer BD Biosciences LSRFortessa™ flow cytometer, BD Biosciences LSRFortess™ X-20 flow cytometer and BD Biosciences FACSCalibur™ cell sorter, a BD Biosciences FACSCount™ cell sorter, BD Biosciences FACSLyric™ cell sorter and BD Biosciences Via™ cell sorter BD Biosciences Influx™ cell sorter, BD Biosciences Jazz™ cell sorter, BD Biosciences Aria™ cell sorters and BD Biosciences FACSMelody™ cell sorter, or the like.

In certain embodiments, the subject systems are flow cytometer systems which incorporate one or more components of the flow cytometers described in U.S. Pat. Nos. 3,960,449; 4,347,935; 4,667,830; 4,704,891; 4,770,992; 5,030,002; 5,040,890; 5,047,321; 5,245,318; 5,317,162; 5,464,581; 5,483,469; 5,602,039; 5,620,842; 5,627,040; 5,643,796; 5,700,692; 6,372,506; 6,809,804; 6,813,017; 6,821,740; 7,129,505; 7,201,875; 7,544,326; 8,140,300; 8,233,146; 8,753,573; 8,975,595; 9,092,034; 9,095,494 and 9,097,640; the disclosures of which are herein incorporated by reference.

Flow cytometric analysis of the sample as described above, yields qualitative and quantitative information about the cells, e.g., monocytes, in the sample. Where desired, the above analysis yields counts of the monocytes of interest in the sample. As such, the above flow cytometric analysis protocol provides data regarding the numbers of one or more different types of monocytes in a sample. The analysis also yields quantitative information about the expression level of HLA-DR in the sample, e.g., in the nature of antibody counts (ABC) per cell.

Where desired, the sample may be subjected to a concentrating step prior to flow cytometric analysis. For example, the sample may be subjected to sample preparation and/or labeling protocols prior to flow cytometric analysis, e.g., such as acoustic and or magnetic manipulation, etc.

As summarized above, the flow cytometric analysis of the labeled sample may include obtaining quantitative expression level data for HLA-DR. The methods may include obtaining the quantitative expression level data for HLA-DR for each monocyte subset present in a sample. The quantitative expression level data may be obtained with an HLA-DR expression level quantitation standard, where the standard may include, e.g., a known quantity of fluorescent labels or fluorescently labeled specific binding members. In some instances, the standard includes one or more particles, e.g., beads, coupled with fluorescent labels or fluorescently labeled specific binding members. The number of fluorescent labels per particle may be known. In some instances, where the particle is coupled to labeled specific binding members, the ratio of fluorescent label to specific binding member is known. The fluorescent labels and/or fluorescently labeled specific binding members present in the HLA-DR expression level quantitation standard may include the same fluorescent labels and/or fluorescently labeled specific binding members used for labeling HLA-DR in the sample.

The methods may include flow cytometrically analyzing the HLA-DR expression level quantitation standard to, e.g., obtain fluorescence intensity level data for the standard. The standard may be flow cytometrically analyzed before or after flow cytometrically analyzing the labeled sample to calibrate the flow cytometric system. The standard and the labeled sample may be analyzed by the same flow cytometric system and may be subjected to the same flow cytometric protocol where one or more flow cytometric parameters, e.g., PE channel PMT voltage, are the same. The fluorescence intensity level data of the HLA-DR expression level quantitation standard may be associated with (e.g., correlated with or indicative of) a known quantity of fluorescent label and/or fluorescently labeled specific binding member. The fluorescence intensity level data that is obtained for the sample may be used to obtain the quantity of fluorescent label and/or fluorescently labeled specific binding member present in a monocyte subset. The quantity of fluorescently labeled specific binding members present in a monocyte subset may be used to determine the expression level of HLA-DR in the monocyte subset. The quantitative expression level data for HLA-DR may include the quantification of HLA-DR expression in antibody bound per cell (ABC) units.

In some instances, the HLA-DR expression level quantitation standard is a composition including BD Quantibrite™ beads. In some instances, obtaining quantitative expression level data for HLA-DR may include obtaining, e.g., calculating, median expression levels of HLA-DR on each monocyte subset in ABC units after calibration of median fluorescence intensity (MFI) in the PE channel using BD Quantibrite™ beads. QuantiBRITE™ PE Beads include a set of four pre-calibrated bead levels in the form of a lyophilized pellet to calibrate the FL2 axis in terms of PE molecules. QuantiQuest is a quantitative calibration feature within CellQuest™ (Becton-Dickinson, San Jose, Calif.) acquisition software (version 3.1 and later) that calculates the linear function relating fluorescence to PE molecules. The PE copy number for a PE-stained cell is determined from the FL2 value of the cell and the linear regression equation.

The number of PE molecules per cell can be converted to antibody bound per cell (ABC) values if the PE:antibody ratio of the antibody conjugate is known. By “antibody bound per cell” or “ABC” is intended the number of a selected antibody of interest that have bound to cells in a selected cell population, e.g., a monocyte subset. In one embodiment, the PE:mAb ratio is 1:1 such that the number of PE molecules per cell is equivalent to the ABC value. The level of antibody binding can be detected using any suitable method.

Having determined the ABC value, the number of antigens per cell can be determined given the antibody-to-antigen binding stoichiometry. Thus, for example, where the antibody-to-antigen binding stoichiometry is 1:1 for a PE-conjugated anti-HLA-DR antibody, the number of HLA-DR antigens is equivalent to the ABC value.

In certain embodiments, the methods further include obtaining cell counts for each identified monocyte subset in the sample, e.g., with a cellular count calibration standard. The sample, e.g., labeled sample, may be combined with the standard, as described above. The labeled sample including the cellular count calibration standard may be flow cytometrically analyzed. The absolute number of cells in a sample may be determined by comparing flow cytometrically detected cellular events to flow cytometrically detected bead events. For example, the absolute count of the cell population (A) may be obtained by dividing the number of positive cell events (X) by the number of bead events (Y), and then multiplying by the BD Trucount™ bead concentration (NN, where N=number of beads per test and V=test volume) (A=X/Y×N/V).

### Compositions

As summarized above, aspects of the present disclosure further include compositions for practicing embodiments of the invention. Compositions suitable for use with the subject methods may include distinguishably fluorescently labeled specific binding members for markers for the identification and characterization of monocyte subsets including, e.g., each of CD14, CD16, CD192 (CCR2) and HLA-DR. The distinguishably fluorescently labeled specific binding members may be present in a composition present in a container. The composition may be present in any suitable container that is compatible with the composition. By “compatible” is meant that the container is substantially inert (e.g., does not significantly react with) the cells, liquid and/or reagent(s) in contact with a surface of the container. Containers of interest may vary and may include but are not limited to a test tube, centrifuge tube, culture tube, falcon tube, microtube, Eppendorf tube, specimen collection container, specimen transport container, and syringe.

In certain embodiments, the composition is a dried composition. A dried composition may be a composition that includes a low amount of solvent. For example, a dried composition may include a low amount of a liquid, such as water. In some cases, a dried composition includes substantially no solvent. For instance, dried compositions may include substantially no liquid, such as water. In certain embodiments, a dried composition includes 25 wt % or less solvent, such as 20 wt % or less, or 15 wt % or less, or 10 wt % or less, or 5 wt % or less, or 3 wt % or less, or 1 wt % or less, or 0.5 wt % or less solvent. In some cases, a dried composition is not a fluid. In some cases, a dried composition is substantially a solid. For example, a dried composition may have a high viscosity, such as a viscosity of 10,000 cP or more, or 25,000 cP or more, or 50,000 cP or more, or 75,000 cP or more, or 100,000 cP or more, or 150,000 cP or more, or 200,000 cP or more, or 250,000 cP or more at standard conditions. In some embodiments, the composition may be present in an amount ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In some embodiments, the composition may be present in an amount ranging from 0.1 g to 10 g, or 0.1 g to 5 g, or 0.1 g to 1 g, or 0.1 g to 0.5 g.

In some instances, the compositions are lyophilized compositions. In certain cases, a lyophilized composition is a composition where water has been removed from the composition by sublimation, where the water in the composition undergoes a phase transition from a solid to a gas. For example, a lyophilized composition may be a composition where water has been removed from the composition by freezing the composition (e.g., freezing water in the composition) and then reducing the pressure surrounding the composition such that the water in the composition undergoes sublimation. In certain instances, a lyophilized composition includes water in a low amount, such as 25% or less, or 20% or less, or 15% or less, or 10% or less, or 9% or less, or 8% or less, or 7% or less, or 6% or less, or 5% or less, or 4% or less, or 3% or less, or 2% or less, or 1% or less, or 0.5% or less, or 0.25% or less, or 0.1% or less water as measured by Karl Fischer (KF) titration. In some cases, a lyophilized composition has 3% or less water as measured by Karl Fischer titration. In some cases, a lyophilized composition has 1% or less water as measured by Karl Fischer titration. In some cases, a lyophilized composition has 0.5% or less water as measured by Karl Fischer titration. Lyophilized compositions may include additives and/or excipients, such as a stabilizer. In some cases, the lyophilized composition includes a stabilizer, such as a sugar or a polyalcohol. Sugars and polyalcohols suitable for use in lyophilized compositions include sugars that are compatible with the other reagents, buffers, and sample components being used. Examples of suitable sugars include, but are not limited to, sucrose, maltose, trehalose, 2-hydroxypropyl-beta-cyclodextrin (β-HPCD), lactose, glucose, fructose, galactose, glucosamine, and the like, and combinations thereof. In certain instances, the sugar is a disaccharide. For example, the disaccharide may be sucrose. Examples of suitable polyalcohols include, but are not limited to, mannitol, glycerol, erythritol, threitol, xylitol, sorbitol, and the like, and combinations thereof.

### Systems

Aspects of the present disclosure further include systems for practicing the subject methods. Systems may include distinguishably fluorescently labeled specific binding members for each of CD14, CD16, CD192 (CCR2) and HLA-DR, e.g., according to any of the embodiments described herein; and a HLA-DR expression level quantitation standard, e.g., according to any of the embodiments described herein. In certain embodiments, the system includes a cellular count calibration standard, e.g., according to any of the embodiments described herein.

The distinguishably fluorescently labeled specific binding members may be present in a container, as described above. In some cases, the distinguishably fluorescently labeled specific binding members may be components of a composition, e.g., a dried composition, present in a container. In some cases, each of the labeled specific binding members includes an antibody or binding fragment thereof.

The HLA-DR expression level quantitation standard may include a composition having a known quantity of fluorescent labels and/or fluorescently labeled specific binding members which may be coupled to one or more particles, e.g., beads. The fluorescent labels and/or fluorescently labeled specific binding members present in the standard may be the same as the fluorescent labels and/or fluorescently labeled specific binding members used for labeling HLA-DR in a sample. Fluorescence intensity level data obtained for the HLA-DR expression level quantitation standard may be associated with a known quantity of fluorescent labels and/or fluorescently labeled specific binding members coupled to one or more particles. The HLA-DR expression level quantitation standard may be, e.g., a BD Quantibrite™ bead composition having the same fluorophore, e.g., PE, as the HLA-DR specific binding member.

In certain embodiments, the systems further include a cellular count calibration standard. The cellular count calibration standard may be used to obtain the absolute cell count for each monocyte subset in a sample. In some cases, the cellular count calibration standard may include a known amount or quantity of detectible particles, e.g., particles bound to a fluorescent label. In some embodiments, the cellular count calibration standard includes a BD Trucount™ bead composition.

### Utility

The subject methods and compositions find use in a variety of applications where identification of monocyte subsets in a sample is desired. Such applications exist in the areas of basic research and diagnostics (e.g., clinical diagnostics). In some instances, the methods include employing an identified monocyte subset, e.g., classical, intermediate, non-classical, in a therapeutic application. Therapeutic applications may vary and include, but are not limited to: predicting therapy responsiveness; evaluating patient responsiveness to a therapy, e.g., immunotherapy, such as anti-PD-1 immunotherapy; diagnosing a disease condition; assessing a disease state, etc. In some instances, the therapeutic application includes evaluating the status of immunosuppression in a subject. In some instances, the absolute count and frequency (%) of intermediate monocytes in a sample may be used in the diagnosis of cardiovascular disease or pre-eclampsia. In some instances, the HLA-DR level and/or frequency (%) of classical monocytes may be used in the diagnosis of sepsis or chronic myelomonocytic leukemia. In some instances, the HLA-DR expression level and/or frequency (%) of classical monocytes may be used to predict therapy efficacy or patient responsiveness to a therapy, e.g., an immunotherapy including, but not limited to, anti-PD-1 therapy.

In certain embodiments, the methods include determining whether the HLA-DR expression level of a monocyte subset, e.g., classical monocytes, is within a predetermined cutoff range. The expression level of HLA-DR, e.g., whether the expression level is within a predetermined cutoff range, may be used to predict patient response to therapy, e.g., immunotherapy. The predetermined cutoff range may range from 5,000 to 20,000 antibody molecules bound per monocyte including, e.g., 5,000 to 15,000 ABC, 5,000 to 10,000 ABC, 10,000 to 20,000 ABC, or 15,000 to 20,000 ABC.

### Kits

Aspects of the present disclosure also include kits. The kits may include, e.g., a container comprising a sample labeling composition, e.g., as described above, such as a reagent composition (which may be dried) that includes distinguishably fluorescently labeled specific binding members for each of CD14, CD16, CD192 (CCR2) and HLA-DR. The kits may further include a cellular count calibration standard, e.g., a BD Trucount™ bead composition. The kits may further include a HLA-DR expression level quantitation standard, e.g., a BD Quantibrite™ bead composition having the same fluorophore, e.g., PE, as the HLA-DR specific binding member. The kits may include a single container or a plurality of containers. Any or all of the kit components may be present in sterile packaging, as desired. Containers of interest may vary and may include but are not limited to a test tube, centrifuge tube, culture tube, falcon tube, microtube, Eppendorf tube, specimen collection container, specimen transport container, and syringe.

The container for holding a component of the kit may hold any suitable volume or quantity of the component. In some cases, the size of the container may depend on the quantity or volume of a component to be held in the container. In certain embodiments, the container may be configured to hold an amount of a component ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of a component ranging from 0.1 g to 10 g, or 0.1 g to 5 g, or 0.1 g to 1 g, or 0.1 g to 0.5 g. In certain instances, the container is configured to hold a volume ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some embodiments, the liquid container may be a vial or a test tube. In certain cases, the liquid container is a vial. In certain cases, the liquid container is a test tube.

Examples of suitable materials for the containers include, but are not limited to, glass and plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

In addition to the above-mentioned components, a subject kit may further include instructions for using the components of the kit, e.g., to practice the subject methods. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., a portable flash drive, CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

The following example provides an embodiment of a method using BD Horizon™ Dri Monoset™ Reagent for identifying monocyte subsets (classical, intermediate, and non-classical) in human whole blood samples by multicolor flow cytometry. Monocyte subset percentages can be measured directly with the panel. When used with BD Trucount™ Tubes, absolute counts of each monocyte subset can be enumerated. Median expression levels of HLA-DR on each monocyte subset can be calculated in antibody bound per cell (ABC) units after calibration of median fluorescence intensity (MFI) in the PE channel using BD Quantibrite™ Beads.

The BD Horizon™ Dri Monoset™ Reagent tube contains a dried pre-aliquoted antibody cocktail containing markers for the identification and characterization of monocyte subsets. The BD Horizon Dri Monoset Reagent is provided as 5 single-use tubes of dried reagent cocktail in a foil pouch. The panel includes the following fluorochrome-conjugated antibodies:

| Specificity | Clone | Fluorochrome |
|---|---|---|
| CD16 | 3G8 | FITC |
| HLA-DR | L243 | PE |
| CD14 | MφP9 | PerCP |
| CD192 (CCR2) | LS132.1D9 | APC |

A whole blood sample is obtained from a subject and prepared for flow cytometric analysis according to the following protocol:
1. Add 50 μl of well-mixed, fresh EDTA-anticoagulated whole blood into the bottom of the dried reagent cocktail tube and
2. Gently pipette/vortex tube for 4 seconds to completely reconstitute the dried antibodies.
3. Incubate 30 minutes at room temperature, protected from exposure to direct light.
4. Add 450 μl of 1×BD FACS Lysing Solution to the tube. Vortex to mix.
5. Incubate for 30 minutes at room temperature, protected from light.
6. Add 1.5 mL of PBS and vortex to mix. The sample is ready to be acquired on a flow cytometer with appropriate compensation settings for FITC, APC, PE and PerCP.

To determine monocyte subset percentages, the sample is subjected to the following protocol:
1. (Optional): To reduce the time needed to acquire the sample,
   a. Centrifuge the tube at 500 g for 5 minutes.
   b. Remove 1.5 mL of the supernatant.
2. Vortex tube thoroughly to resuspend the stained cells.
3. Acquire the sample.

To determine absolute counts of monocyte subsets, the sample is subjected to the following protocol:
1. Transfer 1.5 mL of solution to a BD Trucount™ Tube.
2. Vortex to mix.
3. Acquire the sample.
The volume adjustment has to be factored into the calculation of absolute cell counts.

To measure the median expression levels of HLA-DR on monocyte subsets are (in ABC units), the following protocol is performed:
1. Prepare a tube of BD Quantibrite™ Beads according to the product data sheet.
2. Acquire the beads on the flow cytometer.
3. Vortex the tube of stained cells.
4. Acquire the stained cells.
Acquire the beads and the stained cells using the same PE channel PMT voltage.
5. Calculate the ABC units of the HLA-DR marker on each monocyte subset.

To analyze the monocyte subsets, the flow cytometric data is subjected to the following protocol: 1) Create an SSC-A vs SSC-H dot plot and draw a gate around the singlet population. 2) Create a CD14 PerCP-A vs SSC-A dot plot and apply the SSC singlet gate. Draw a gate to encompass the SSClo populations (CD14+ and CD14+/−). 3) Create an FSC-A vs SSC-A dot plot and apply the CD14 gate. Draw a gate around the population with high forward scatter. 4) Create a CD14 PerCP-A vs HLA-DR PE dot plot and apply the FSC gate. Draw a gate to encompass HLA-DR+ cells. This is the monocyte population. 5) Create a CD14 PerCP-A vs CD16 FITC dot plot and apply the HLA-DR/monocyte gate. Draw two gates, the first gate encompassing CD14+CD16lo cells (classical (M1) monocytes), and the second gate encompassing CD16+ cells. 6) Create a CCR2(CD192) APC-A vs SSC-A dot plot and apply the CD16+ monocyte gate. Draw two gates, the first gate encompassing CD192 hi cells (intermediate (M2) monocytes, and the second gate encompassing CD192lo cells (non-classical (M3) monocytes. 7) Calculate relative percentages of monocyte subsets from the cell counts.

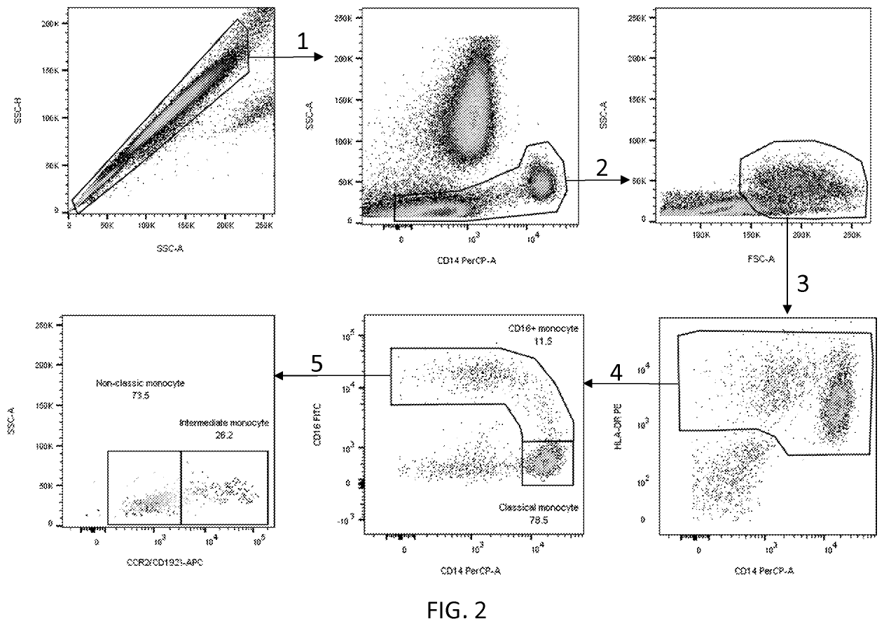

FIG. 2 provides an example of gating for monocyte subsets using data acquired on a BD FACSLyric™ flow cytometer and analyzed using BD FlowJo™ software.

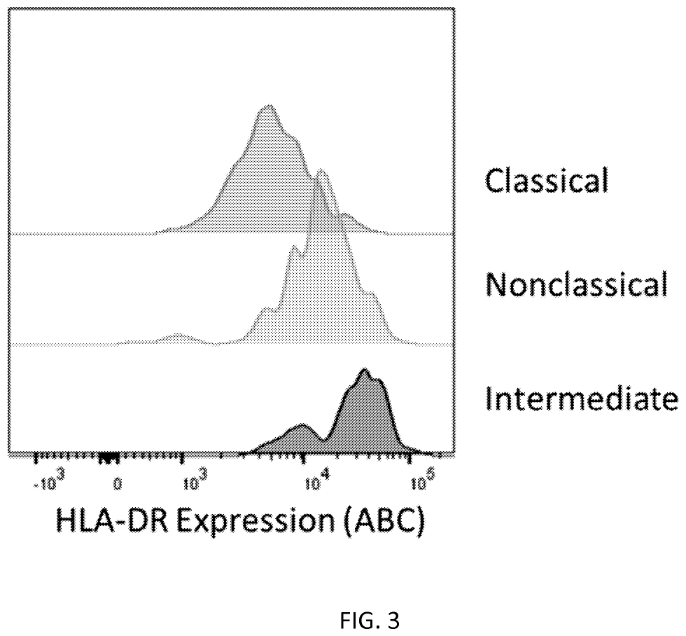

FIG. 3 shows HLA-DR expression for monocyte subsets in antibody bound per cell units (ABC).

To identify the Trucount™ Beads population, the flow cytometric data is subjected to the following protocol: 1) Create an FSC-A vs SSC-A and draw two gates, the first gate encompassing the BD Trucount™ beads population, and the second gate encompassing the cell population. 2) Create an HLA-DR PE-A vs CD16 FITC-A dot plot and apply the Trucount™ beads gate. Draw a gate to encompass the beads population. 3) Analyze the cell population, identified in the first dot plot, using the gating strategy described previously. 4) Calculate the absolute count of the desired population using the following equation:

$$\frac{\# events in cell population}{\# events in Trucount beads region} \times \frac{\# beads / test}{test volume ( μL )} = cell population absolete count ( cells / μL )$$

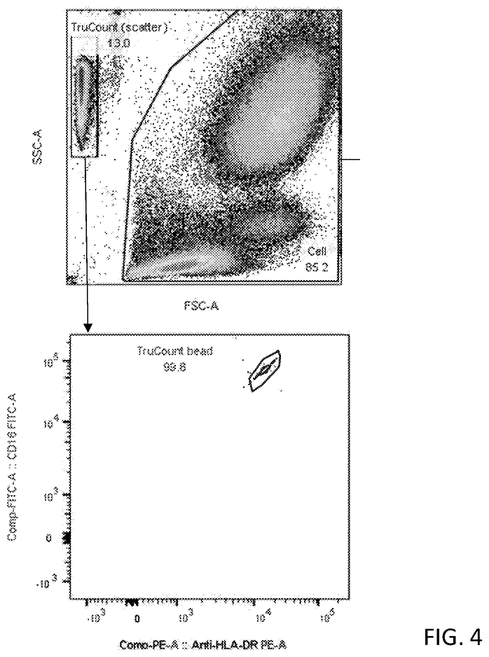

FIG. 4 provides an example of a BD Trucount™ beads gating strategy.

A comparison of results obtained from whole blood samples of healthy donors using the dried four-color reagent panel and results obtained using the liquid cocktail is shown in Table 1. An example of monocyte subset analysis using the dried four-color reagent panel with BD TruCount™ and BD Quantibrite™ tubes is shown in Table 2.

TABLE 1
Percentages of monocyte subsets relative to total monocytes and HLA-DR expression
levels (in ABC units after calibration with BD Quantibrite beads) for five
healthy donors using whole blood samples stained with the four-color dried
reagent panel and liquid cocktail of the same reagents.

| | | Monocyte Subset (%) | | | Monocyte Subset HLA-DR (ABC) | | |
|---|---|---|---|---|---|---|---|
| Donor | Reagent | M1 | M2 | M3 | M1 | M2 | M3 |
| D1 | Dried | 88.7% | 3.1% | 8.2% | 19,990 | 126,055 | 34,681 |
| D1 | Liquid | 87.4% | 3.7% | 8.8% | 18,839 | 110,754 | 32,609 |
| D2 | Dried | 89.4% | 4.1% | 6.5% | 27,358 | 185,005 | 65,096 |
| D2 | Liquid | 88.3% | 5.2% | 6.5% | 23,643 | 160,884 | 62,814 |
| D3 | Dried | 86.8% | 4.5% | 8.6% | 14,792 | 131,503 | 39,864 |
| D3 | Liquid | 86.8% | 4.7% | 8.6% | 14,051 | 129,735 | 38,254 |
| D4 | Dried | 92.9% | 3.2% | 3.8% | 14,583 | 140,314 | 51,390 |
| D4 | Liquid | 92.2% | 3.4% | 4.4% | 14,424 | 137,055 | 48,958 |
| D5 | Dried | 86.0% | 4.2% | 9.8% | 16,376 | 149,415 | 47,602 |
| D5 | Liquid | 87.1% | 3.4% | 9.4% | 15,497 | 163,866 | 44,144 |

TABLE 2
Example of monocyte subset analysis using dried reagent tubes with BD TruCount ™ and BD
Quantibrite ™ tubes. Absolute count, relative frequency and HLA-DR expression (in antibody
bound per cell units) values for each subset of monocytes were obtained health donor whole blood samples.

| | | | | | | | Monocyte Subset HLA-DR | | |
|---|---|---|---|---|---|---|---|---|---|
| | Absolute Count/μl | | | Monocyte Subset (%) | | | (ABC) | | |
| | | | Non- | | | Non- | | | Non- |
| Donor | Classical | Intermediate | classical | Classical | Intermediate | classical | Classical | Intermediate | classical |
| D1 | 469 | 21 | 33 | 89.7% | 4.0% | 6.2% | 15,750 | 100,025 | 30,884 |
| D2 | 307 | 10 | 77 | 78.0% | 2.4% | 19.5% | 13,460 | 132,536 | 41,316 |
| D3 | 355 | 29 | 58 | 80.3% | 6.7% | 13.0% | 16,426 | 99,792 | 38,576 |
| D4 | 424 | 30 | 51 | 84.0% | 6.0% | 10.0% | 20,779 | 126,989 | 49,072 |
| D5 | 341 | 12 | 37 | 87.4% | 3.0% | 9.5% | 15,502 | 107,601 | 40,860 |
| D6 | 418 | 19 | 49 | 86.1% | 3.8% | 10.0% | 19,822 | 117,153 | 41,405 |

### Example 2

An exemplary method for identifying monocyte subsets in a sample is provided. The method provides for measuring the percentage of each subset of monocytes (classical, intermediate and nonclassical) and quantifying the HLA-DR level on each subset based on measurement of antibody bound per cell (ABC) using BD Quantibrite™ beads. Results were obtained with dried reagents and liquid reagents.

### Reagent Preparation:

Reagent Panel

| | | | | | | Assay |
|---|---|---|---|---|---|---|
| | | | bulk conc. | ug/test | | conc. |
| Reagent | Cat. number (BD) | Lot number | (mg/mL) | in assay | volum/test | (mg/mL) |
| CD192-APC | PC2816 (custom) | (custom) | 0.26 | 0.25 | 10 | 0.025 |
| HLA-DR-PE | 50-7897 | 8155801 | 0.453 | 1 | 10 | 0.1 |
| CD14-PerCP | 50-00210 | 8088953 | 0.351 | 1 | 10 | 0.1 |
| CD16-FITC | 51-54030624 | 7340744 | 0.519 | 0.5 | 10 | 0.05 |

Prepare reagent mix according to the concentration listed in the table above. For dried tubes, add 10 uL to each tube and let dried with ~37 °C air for 10 min. Place 5 tubes in each alumina pouch with desiccant and store at room temperature for at least 16 hours prior to use. Store the reminder of reagent at 4 °C.

### Staining and Testing

### Staining

1) To each tube with dried reagent: add 50 μL of whole blood to each tube and mix.
2) Liquid reagent testing: add 10 μL of reagent mix into each tube; add 50 μL of whole blood to the tube and mix.
3) Incubate the tubes at room temperature for 30 min and protect the tubes from light.
Add 450 μL of 1×FACSLyse to each tube and incubate in dark for 15 min.
Add 1500 μL of PBS buffer to each tube.
Analyze cells on BD FACSLyric based on a lyse/wash setting.

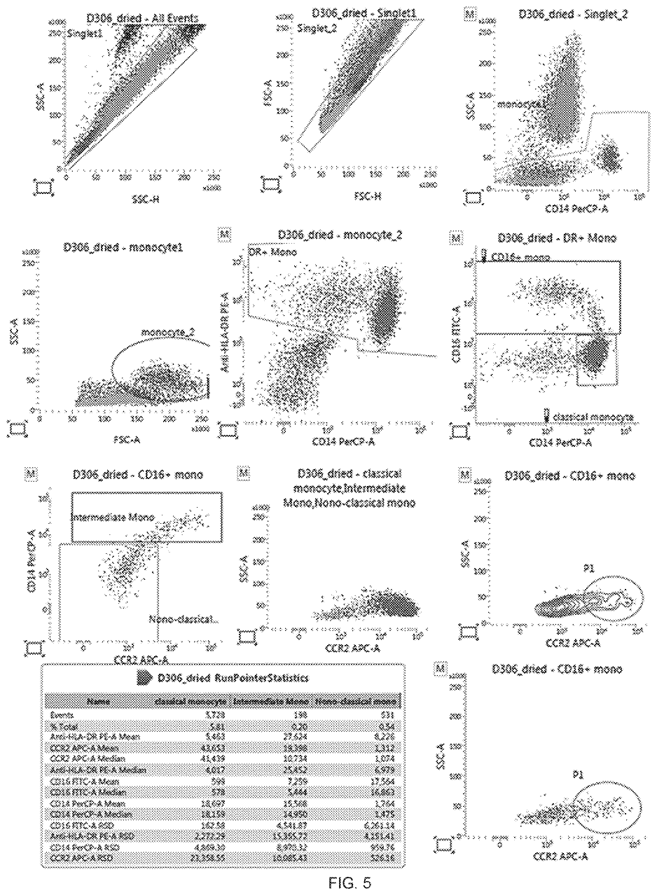

FIG. 5 shows exemplary gating for monocyte subset analysis in whole blood using a four-reagent panel (CD192, HLA-DR, CD14, CD16). The flow cytometric data is obtained with the BD FACSLyric™ system.

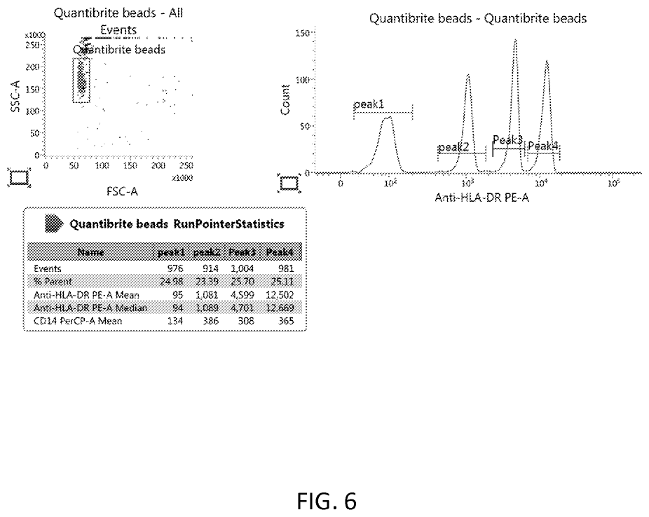

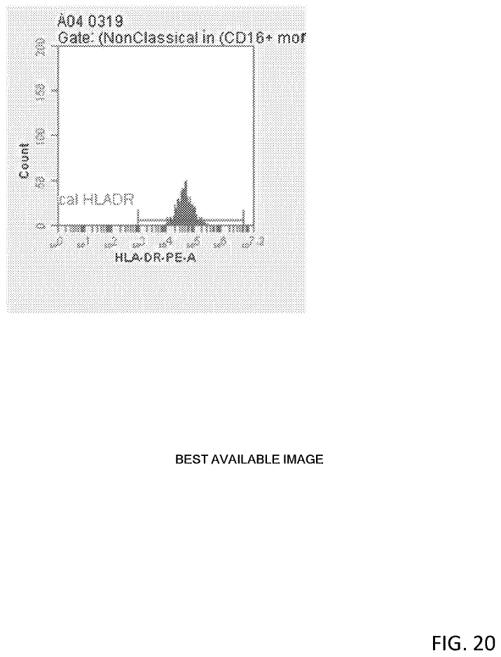

FIG. 6 shows BD Quantibrite™ bead analysis. The beads are analyzed under the same flow cytometric conditions as applied to the whole blood sample in FIG. 20.

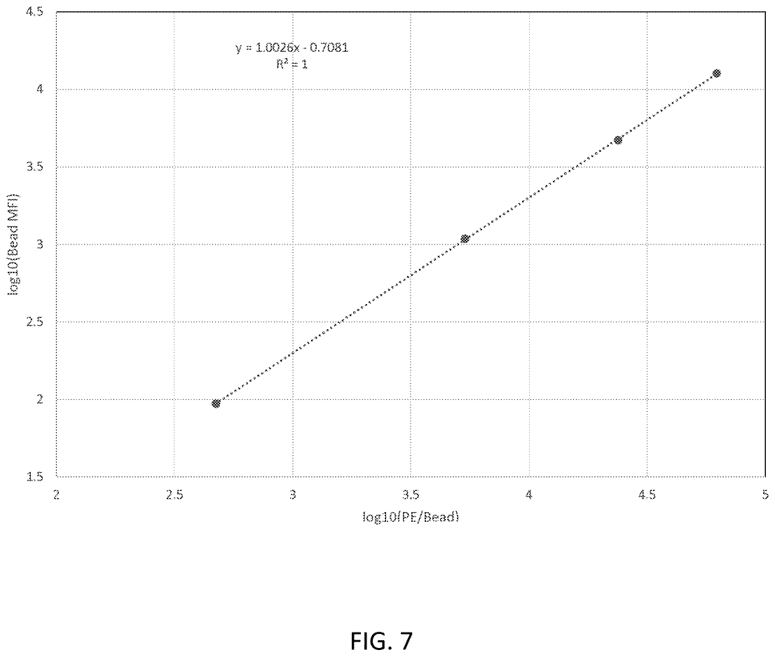

FIG. 7 shows BD Quantibrite™ bead analysis and calibration. For cells, ABC values=10\hat{ }((LOG 10(MFI)−intercept)/slope)=10\hat{ }((LOG 10(MFI)+0.7081)/1.0026)*MFI is the median fluorescence intensity of the PE channel measured for the cells stained with HLA-DR PE.

### Example 3

An exemplary method for identifying monocyte subsets in a whole blood sample is provided where the method includes the following steps:
a) Staining a whole blood sample from a patient using a reagent panel comprising fluorescently labeled antibodies against CD14, CD16, HLA-DR and CCR2 biomarkers, with each biomarker stained with a different fluorescence color.
b) Analyzing the stained cells on a flow cytometer and gate classic, intermediate and non-classic subsets of monocytes based on the fluorescence intensity levels and forward and side scattering parameters. Measure the mean or median fluorescence intensity of the HLA-DR biomarker on each subset of monocytes.
c) Analyzing a calibration bead sample containing a fluorescence dye that is identical to the fluorescence dye used for labeling the HLA-DR biomarker, with at least one known level of the number of fluorescence dye molecules per bead. Calibrate the fluorescence intensity measured on the beads with the known level of fluorescence dye molecules per bead.
d) Based on the calibration of the fluorescence intensity measured in step 3 and the fluorescence intensity for each subset of monocytes measured in step 2, calculate the HLA-DR levels based on the number of antibody molecules bound on each of the monocyte subsets.

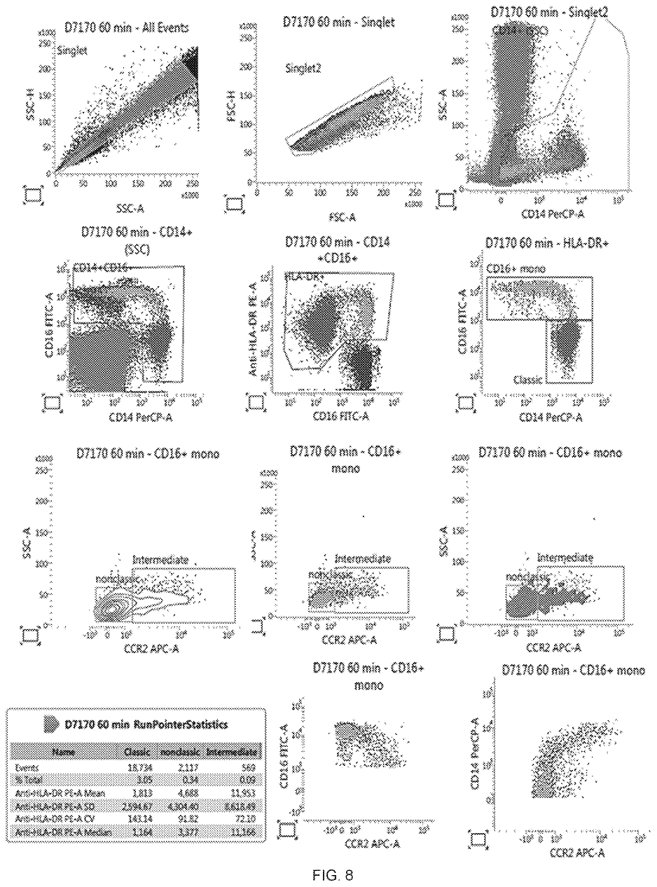

FIG. 8 shows exemplary gating for monocyte subset analysis in whole blood using a four-reagent panel (CD192, HLA-DR, CD14, CD16). The flow cytometric data is obtained with the BD FACSVia™ system.

### Example 4

Exemplary HLA-DR expression level data for monocyte subsets obtained from subjects with the FACSVIA™ system are provided in Table 3.

TABLE 3
FACSVia ™ Results Summary (5-donor; 50 μl whole blood stained
with CD14/CD16/HLA-DR/CCR2 for 30 min; Lyse no wash).

| | Classical HLADR | | | | Intermediate HLADR | | |
|---|---|---|---|---|---|---|---|
| | | Mean | CV | Median | | Mean | CV |
| Donor# | Count | FL2-A | FL2-A | FL2-A | Count | FL2-A | FL2-A |
| A05 | 7,726 | 36,801.09 | 128.23% | 24,754.00 | 165 | 273,996.66 | 52.69% |
| A01 | 9,297 | 36,024.37 | 136.55% | 22,366.00 | 187 | 247,681.12 | 61.28% |
| A02 | 7,347 | 40,457.99 | 134.28% | 24,751.00 | 328 | 216,554.82 | 57.06% |
| A03 | 2,257 | 28,166.03 | 129.82% | 17,736.00 | 118 | 145,075.08 | 99.17% |
| A04 | 6,461 | 29,512.00 | 145.67% | 16,809.00 | 287 | 202,245.71 | 63.20% |

| | Intermediate HLADR | non-classical HLADR | | | |
|---|---|---|---|---|---|
| | Median | | Mean | CV | Median |
| Donor# | FL2-A | Count | FL2-A | FL2-A | FL2-A |
| A05 | 253,845.00 | 370 | 89,114.43 | 80.61% | 70,418.50 |
| A01 | 236,821.00 | 323 | 96,230.82 | 82.89% | 74,292.00 |
| A02 | 200,926.00 | 683 | 96,266,73 | 85.76% | 71,277.00 |
| A03 | 109,30.50 | 360 | 40,719.59 | 76.55% | 30,757.00 |
| A04 | 189,981.00 | 649 | 66,954.94 | 99.50% | 49,751.00 |

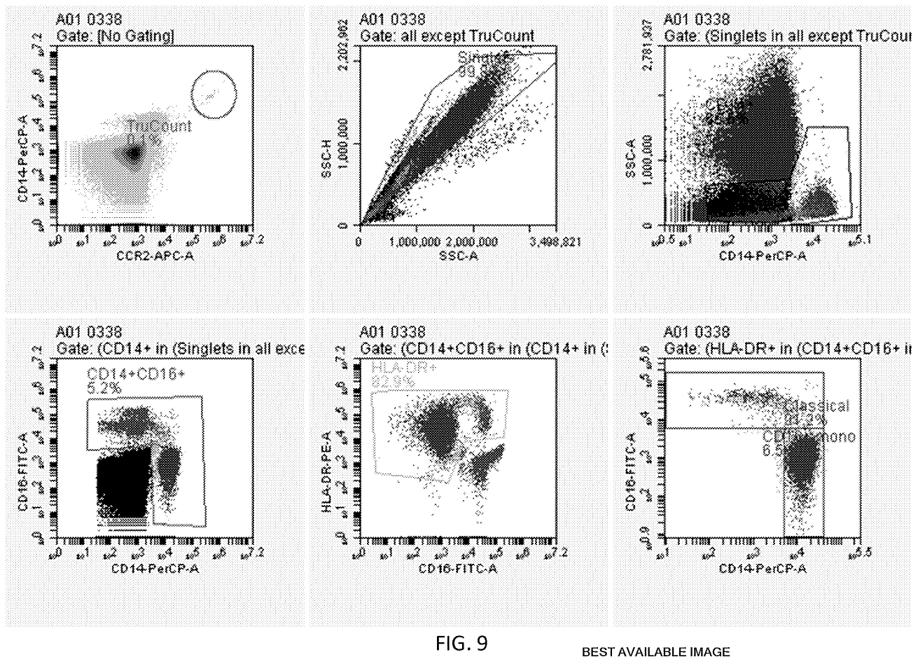

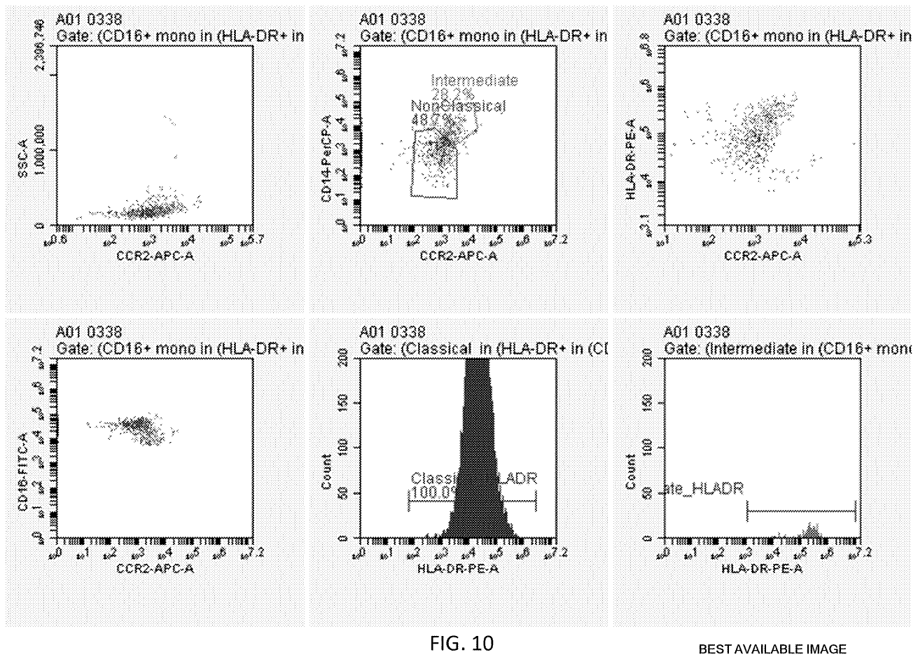

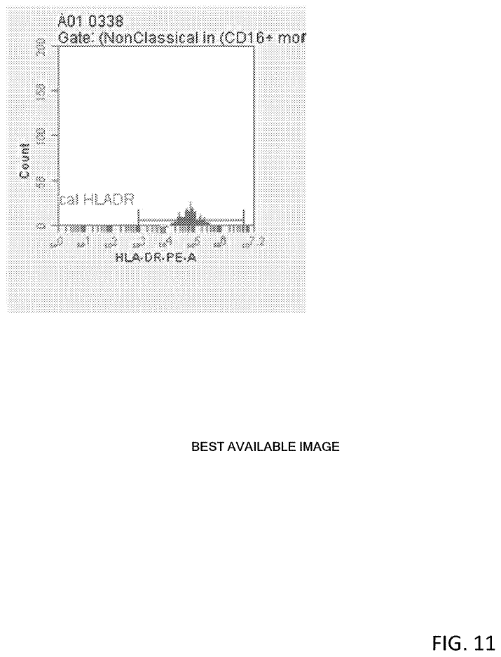

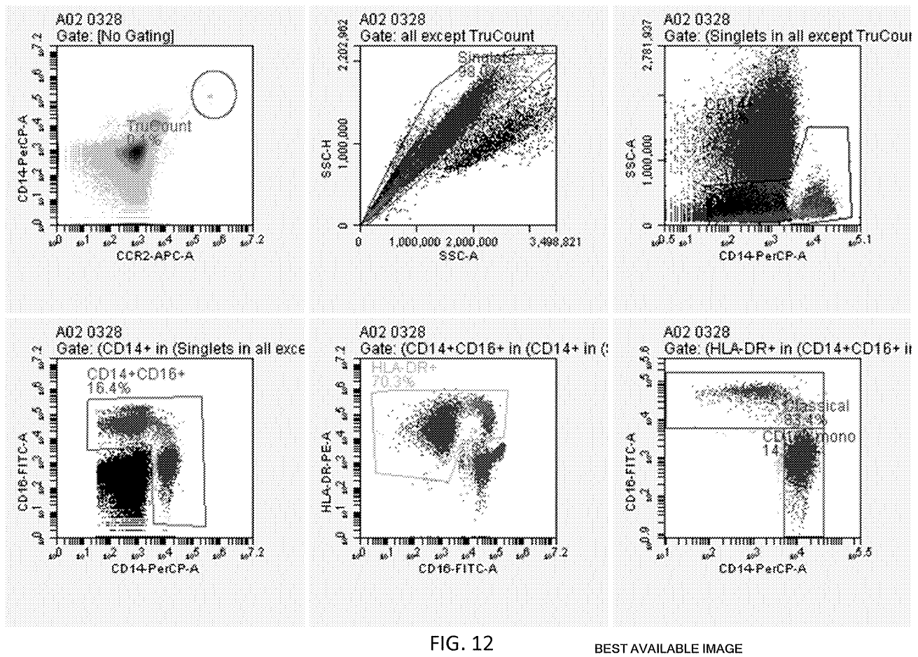

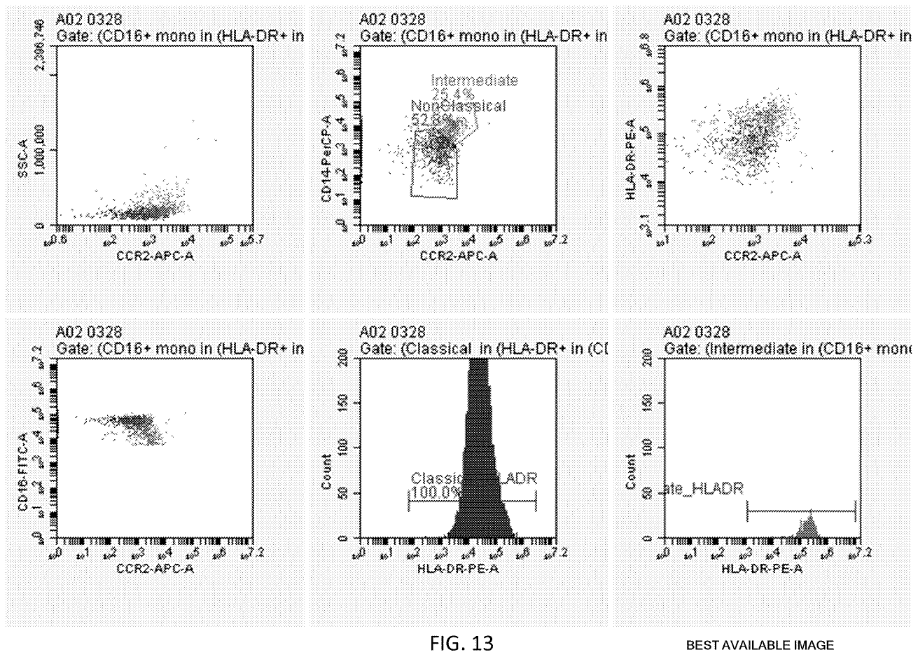

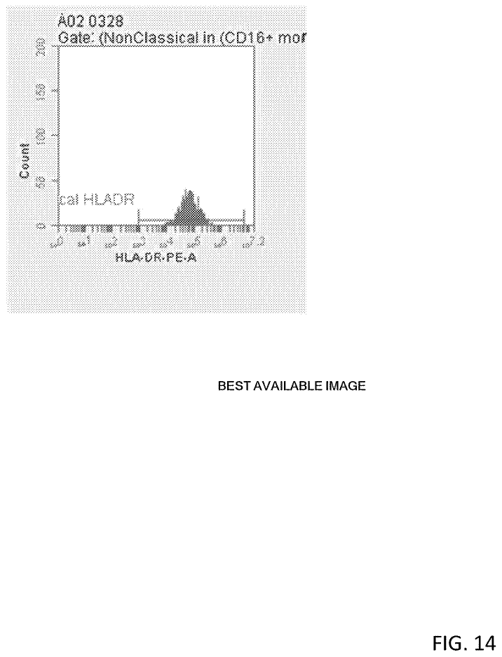

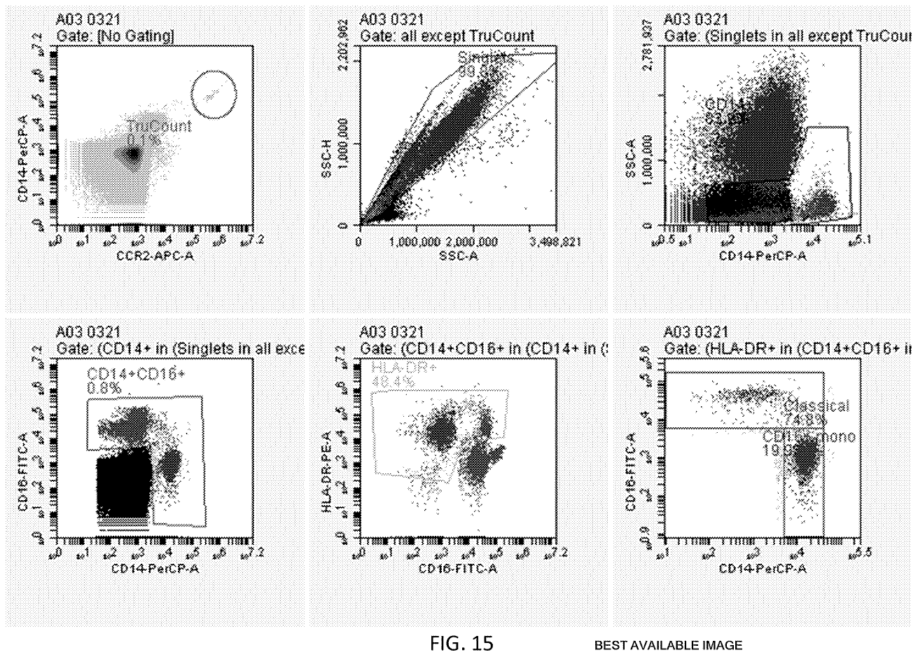

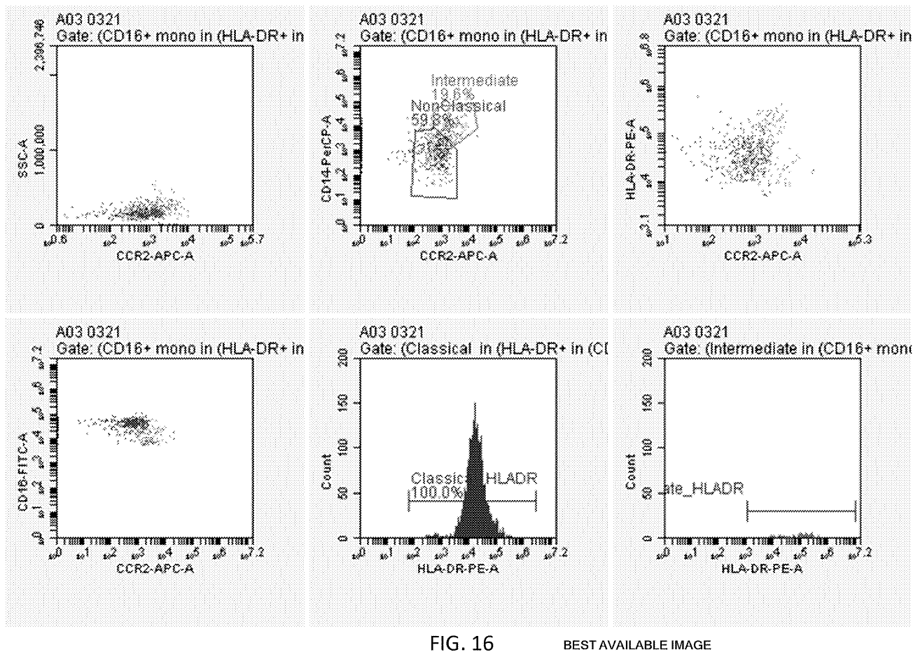

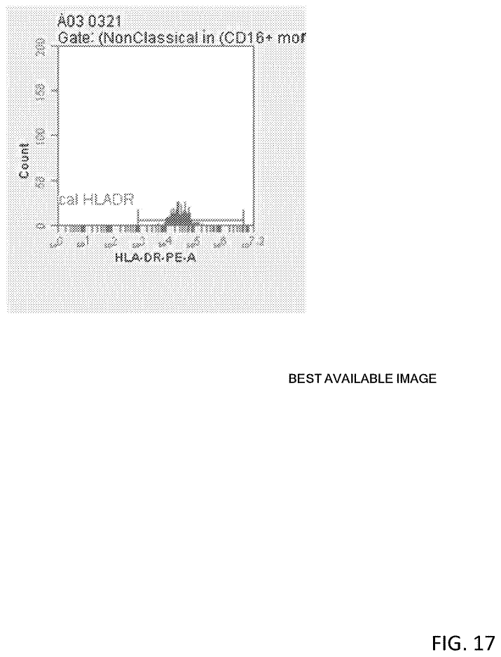

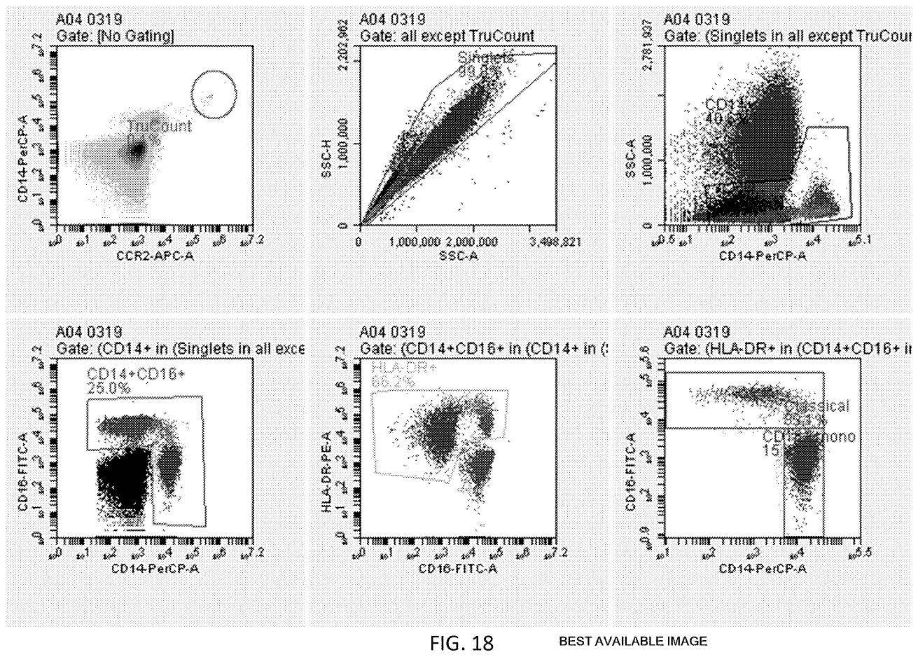

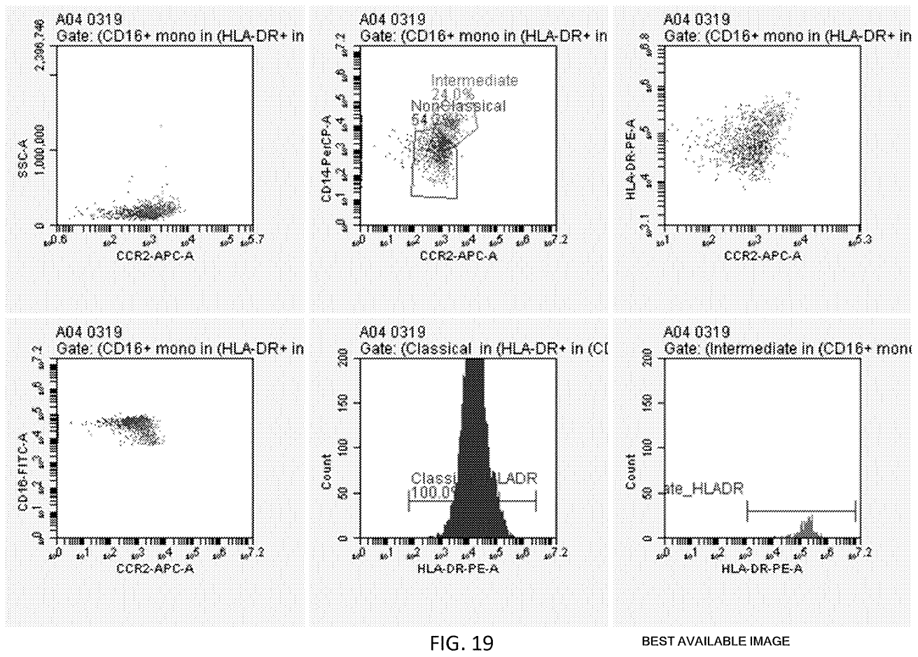

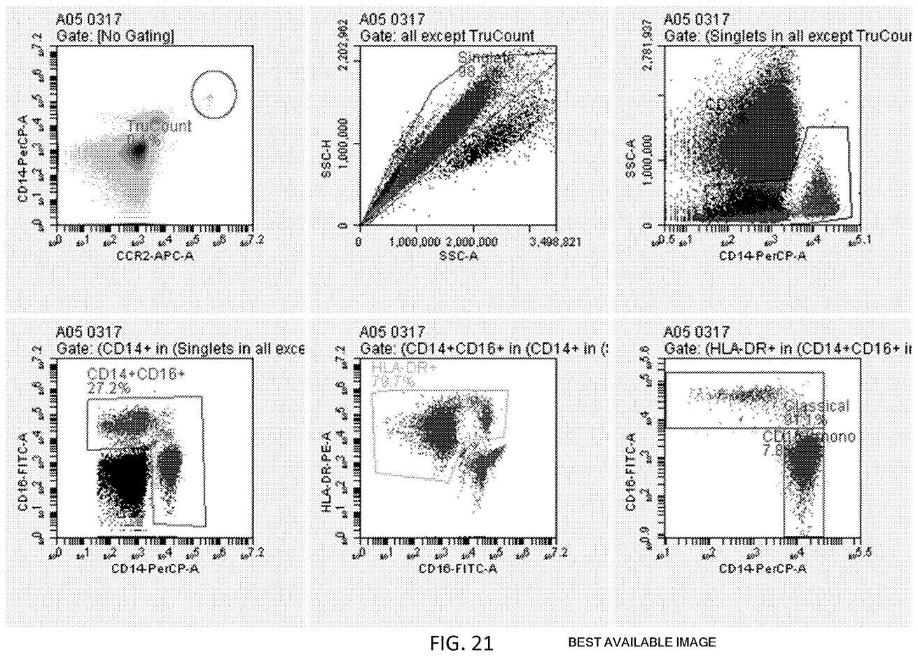

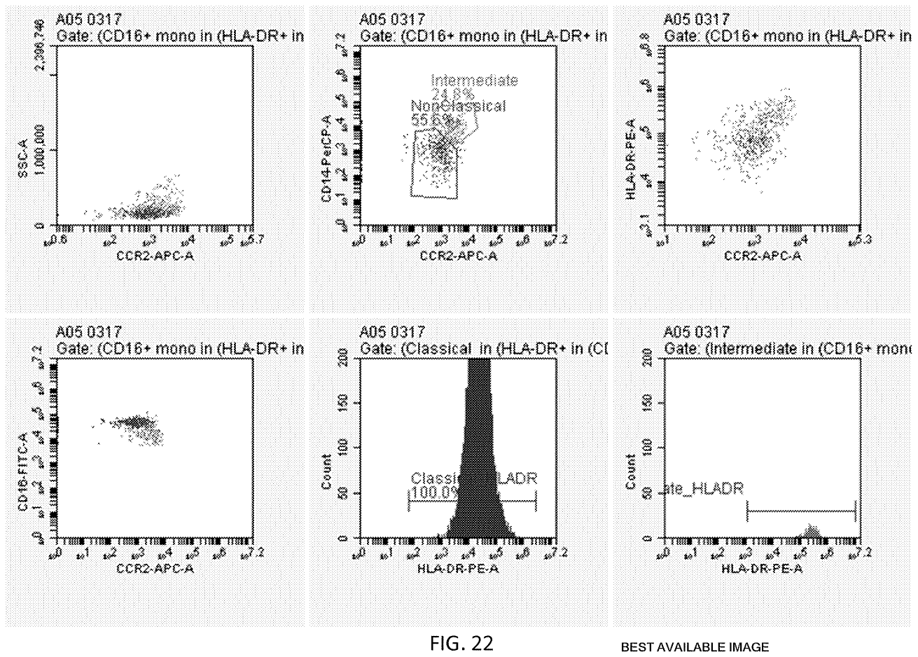

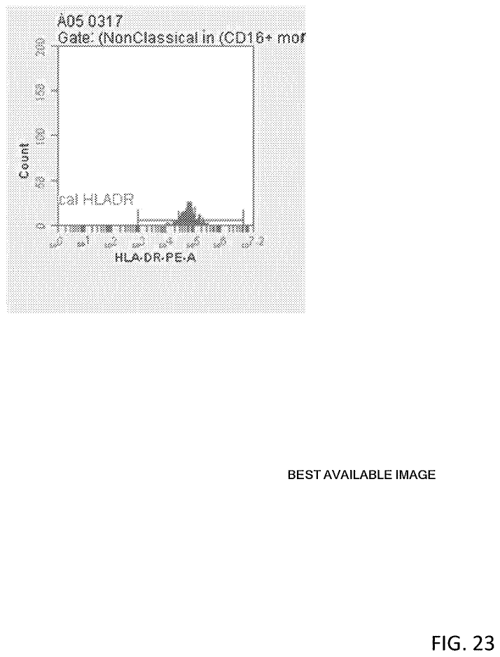

FIGS. 9-23 show gating for monocyte subsets and HLA-DR analysis in samples obtained from the five donors represented in Table 1 (A01: FIGS. 9-11; A02: 12-14; A03:15-17; A04: FIGS. 18-20; A05: FIGS. 21-23).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-20. (canceled)

21. A kit comprising:
distinguishably fluorescently labeled specific binding members for each of CD14, CD16, CD192 (CCR2) and HLA-DR; and
a HLA-DR expression level quantitation standard.

22. The kit according to claim 21, wherein each of the labeled specific binding members comprises antibody or binding fragment thereof.

23. The kit according to claim 21, wherein the labeled specific binding members are present in a container.

24. The kit according to claim 23, wherein the labeled specific binding members are dried.

25. The kit according to claim 21, wherein the HLA-DR expression level quantitation standard comprises beads labeled with the same fluorescent label as that of the HLA-DR specific binding member.

26. The kit according to claim 21, further comprising a cellular count calibration standard.

27. A composition comprising distinguishably fluorescently labeled specific binding members for each of CD14, CD16, CD192 (CCR2) and HLA-DR.

28. The composition according to claim 27, wherein the composition is present in a container.

29. The composition according to claim 28, wherein the container comprises a tube.

30. The composition according to claim 29, wherein the composition is dried.

31. The composition according to claim 27, wherein each of the labeled specific binding members comprises antibody or binding fragment thereof.

32. A container having present therein a composition comprising distinguishably fluorescently labeled specific binding members for each of CD14, CD16, CD192 (CCR2) and HLA-DR.

33. The container according to claim 32, wherein each of the labeled specific binding members comprises antibody or binding fragment thereof.

34. The container according to claim 32, wherein the container comprises a tube.

35. The container according to claim 32, wherein the composition is dried.
